(19) United States
(12) Patent Application Publication
(10) Pub. No.: US 2004/0043071 A1
(43) Pub. Date: Mar. 4, 2004

(54) Intravaginal mucosal or transmucosal delivery of antimigraine and antinausea drugs

(76) Inventors: Giovanni M. Pauletti, Loveland, OH (US); Richard Soderstrom, Seattle, WA (US); Wolfgang A. Ritschel, Cincinnati, OH (US)

Correspondence Address: Hana Verny, Peters, Verny, Jones & Schmitt, LLP, Suite 6, 385 Sherman Avenue, Palo Alto, CA, 94306, US

(21) Appl. No.: 10/600,849

(22) Filed: Jun. 20, 2003

Related U.S. Application Data
(60) Provisional application No. 60/390,748, filed on Jun. 21, 2002.

(51) Int. Cl. A61K 9/14

(52) U.S. Cl. 424/484

(57) ABSTRACT

A method, composition and device for intravaginal mucosal or transmucosal delivery of antimigraine and/or antinausea drugs to a female subject for treatment of migraine and other diseases accompanied by or associated with nausea and vomiting. A mucoadhesive composition comprising antimigraine or antinausea drugs, mucoadhesive agent, penetration enhancer or sorption promoter and a hydrophilic or lipophilic carries. An intravaginal device for delivery of antimigraine or antinausea drugs.

This application is based on and claims priority of the Provisional Application Ser. No. 60/390,748 filed on Jun. 21, 2002.

### BACKGROUND OF THE INVENTION

### FIELD OF THE INVENTION

The present invention concerns a method, composition and device for intravaginal mucosal or transmucosal delivery of antimigraine and/or antinausea drugs to a female subject for treatment of migraine and other diseases accompanied by or associated with nausea and vomiting. In particular, the invention concerns a method, composition, and device for mucosal delivery of antimigraine and/or antinausea drugs to the vagina for topical vaginal treatment or for transmucosal delivery of these drugs into the systemic blood circulation for systemic therapy using a mucoadhesive composition comprising these drugs. The composition is administered directly or incorporated into an intravaginal device.

The mucoadhesive composition of the invention or intravaginal device incorporated with said composition delivers the antimigraine and/or antinausea drug into the vagina, provides a continuous contact with the vaginal mucosa, releases the therapeutic agent from the formulation in timely fashion and at controllable quantities, and delivers the drugs transmucosally across the vaginal epithelial barrier into the systemic circulation. The mucoadhesive composition adheres to the vaginal mucosa and promotes topical adhesion of the drug released from said composition to the vaginal mucosa and further promotes delivery of the drug transmucosally through the vaginal mucosa and wall to the systemic circulation.

The method of the invention for treatment of migraine, nausea and vomiting eliminates need for oral administration and avoids its passage through the gastrointestinal tract thereby preventing extensive first-pass metabolism of the therapeutic antimigraine and/or antinausea agent by the liver and further permits efficacious, rapid, continuous or pulsed delivery of the antimigraine and/or antinausea drugs resulting in delivery of therapeutically effective concentrations of such agents to the female subject.

### BACKGROUND AND RELATED DISCLOSURES

Migraine is a common illness, which imposes an enormous health burden on both patient and society. In the United States alone, it is estimated that between 25 to 30 million people experience this condition leading to significant work and productivity loss.

Migraine is a chronic condition with recurrent episodic attacks. It is rather unpredictable illness with its characteristics varying among patients. This unpredictability and variability is also observed within migraine attacks observed in a single patient. Among the most distinguishing features of a migraine is a potential disability caused by the accompanying headache and nausea with or without vomiting as well as extreme sensitivity to sound and light (Headache, 39: 720-727 (1999)). Because of the variability and complexity of the condition, effective management of patients suffering from migraines is challenging.

Migraine headaches which are considered “primary headaches” are about three times more common in women than in men. Geographically, the occurrence of migraine headaches varies significantly and ranges from 1.5% in Southeast Asia to 14% in Western countries (GRIM, Cephalalgia, 12:229 (1992); JAMA, 267:64-69 (1992); and Pharmacoeconomics, 11: 1-10 (Suppl.1)(1997)).

In childhood, the number of boys and girls suffering from migraine is similar, however, during the adolescent period, the number of females who are afflicted by this condition increases steadily throughout early adulthood. The difference between males and females reaches its peak when patients are in their 40's. The female/male gender difference in migraine frequency declines following menopause suggesting the female hormones have an important influence on migraine.

By the majority of migraine sufferers, migraines begin or are felt on one side of the head and the pain is typically throbbing in nature. In about 20% of all migraine cases, an aura—a group of neurological symptoms—occurs before the head pain begins. Typically, an aura involves a disturbance in vision that may consist of bright colored or blinking lights in a pattern that moves across the field vision. Typically, migraine attacks are occasional and are unpredictable. Sometimes these attacks may occur as often as once a week, but usually they do not occur daily.

Effective pharmacotherapy of migraine includes various drug classes that alone or in combination with other drugs provide relief from the headache pain and, in addition, reduce associated symptoms such as nausea and vomiting. Although generally the oral route for drug delivery is the most preferred route of administration for over 80% of approved drug products because of its ease and effectiveness, this route of administration is not the most effective for treatment of migraine patients.

It is generally known and accepted that immediately prior or during a migraine attack, oral administration of drug will stimulate vomiting of a patient who may already suffer from migraine-associated nausea. If the patient is able to swallow a drug orally despite nausea, subsequent vomiting which almost always follows dramatically reduces the time available for the drug to be effective for migraine treatment. Consequently, a limited time during which the drug is present in the gastrointestinal tract permits only incomplete delivery of the desired therapeutic dose and the efficacy of the treatment is severely compromised. In addition, the unpredictable delay in the onset of vomiting following administration of the oral dosage form to migraine patients leads to significant variability in symptom relief.

Common alternative routes of administration that bypass these problems include parenteral injection (e.g., intramuscular, subcutaneous), nasal spray, or administration of a rectal dosage form. Injection methods usually require assistance of a trained health care professional whereas many patients find insertion of a rectal dosage form uncomfortable and/or emotionally unpleasant. Nasal delivery systems for migraine therapy have been successful but remain limited to drugs that experience only minimum hepatic metabolism.

Transvaginal delivery of anti-inflammatory and other drugs using a vaginal device has been discovered by inventors and is disclosed in the U.S. Pat. Nos. 6,086,909; 6,197,327; 6,416,779; and 6,572,874.

Thus, it would be advantageous to have available an alternative route of administration that would eliminate nausea and vomiting connected with migraines, which would overcome the negative impact of vomiting on the delivered dose and also the limitations associated with parenteral injections, nasal administration, and insertion of rectal dosage forms. Furthermore, a therapeutically desired alternative treatment should also achieve reproducible and complete delivery of drugs that effectively control symptoms of migraine and nausea.

It is, therefore, a primary objective of this invention to provide an alternative route for delivery of anti-migraine and antinausea agents which route would eliminate complications connected with oral administration of these drugs. This objective is met by a discovery that an appropriately formulated mucoadhesive composition delivered alone or incorporated into a vaginal device provides an effective method for administration of antimigraine and/or antinausea drugs by transmucosal or topical vaginal delivery resulting in efficacious treatment of migraine, nausea and vomiting.

All references, patents and patent applications cited herein are hereby incorporated by reference in their entirety.

### SUMMARY OF THE INVENTION

One aspect of the current invention is a method, composition, and a device for vaginal delivery of effective doses of an antimigraine and/or antinausea drug to the vaginal mucosa or transmucosally to the general blood circulation.

Another aspect of the current invention is a method for intravaginal mucosal or transmucosal delivery of antimigraine and/or antinausea drugs to a female subject for treatment of migraine and a method for treatment of other diseases and conditions accompanied by or associated with headaches, nausea and vomiting.

Yet another aspect of the current invention is a method, composition, and device for mucosal and transmucosal delivery of antimigraine and/or antinausea drugs to the vagina for topical vaginal treatment and/or for transmucosal delivery of these drugs into the systemic blood circulation for systemic therapy using a mucoadhesive composition comprising these drugs directly or incorporated into an intravaginal device.

Another aspect of the current invention is a method for treatment, management, and control of migraine and headache pain, nausea, and vomiting associated with migraine or other conditions, such as following the surgery, radiotherapy or administration of chemotherapeutic drugs, said method comprising steps of contacting vaginal mucosa with a mucoadhesive composition or with an intravaginal device incorporated with said mucoadhesive composition, said composition comprising an antimigraine agent selected from the group consisting of ergot alkaloids and derivatives, antihistamines, barbiturates, non-steroidal anti-inflammatory agents, analgesics, serotonin antagonists, neurokinin-1 antagonists, cannabinoids, calcitonin gene-related peptide (CGRP) antagonists, steroids, sympathomimetics, tranquilizers and antiepileptics each alone or in combination with an antinausea drug or further in combination with other pharmaceutical agents or pharmaceutically acceptable excipients and maintaining said composition or device in contact with said vaginal mucosa for a period of time permitting a rapid, continuous or pulsed delivery of the agent to or through the vaginal mucosa, said composition further comprising at least a mucoadhesive agent, lipophilic or hydrophilic carrier and permeation enhancer.

Still another aspect of this invention is a method for treating a human female subject suffering from migraine or headache and/or nausea and vomiting associated with migraine or other disease or conditions, said method comprising steps of contacting vaginal mucosa with a mucoadhesive composition or with an intravaginal device incorporated with said composition, said mucoadhesive composition comprising an antimigraine drug selected from but not limited to the group of compounds consisting of ergotamine, dihydroergotamine, ergostine, butalbital, phenobarbital, acetaminophen, diclofenac sodium, ketoprofen, ketorolac, ibuprofen, prioxicam, naproxen, acetylsalicilic acid, flurbiprofen, tolfenamic acid, butorphanol, meperidine, methadone, sumatriptan, naratriptan, razatriptan, zolmitriptan, almotriptan, eletriptan, dexamethasone, hydrocortisone, isometheptene, chlorpromazine, diazepam, droperidol, valproic acid, gabapentin, topiramate, divalproex sodium, or antinausea drug metoclopramide, prochlorperazine, domperidone, ondansetron, tropisetron, dolasetron, nabilone, dronabinol, levonantradol, CP55,940, SR 144528, aprepitant, cyclizine, and promethazine, BIBN-4096BS, SB-(+)-273779, alone or in combination with an another pharmaceutical agent or a pharmaceutically acceptable excipient, said composition further compromises at least a mucoadhesive agent, a lipophilic or hydrophilic carrier and a permeation enhancer.

Still another aspect of this invention is a mucosal composition comprising an antimigraine and/or antinausea drug alone or in admixture with another pharmaceutical agent or a pharmaceutically acceptable excipient, said composition suitable for administration to the vagina or for incorporation into an intravaginal device for the vaginal or transmucosal vaginal delivery of the drug through the vaginal mucosa into the systemic blood circulation, said agent present in an amount sufficient to assert its therapeutic effect.

Still yet another aspect of this invention is a mucoadhesive composition for mucosal or transmucosal delivery of antimigraine and/or antinausea drugs said composition comprising, in dosage unit form, from 0.001 to 3000 mg, preferably from 0.1 to 1000 mg, of an antimigraine agent selected from the group consisting of but not limited to ergotamine, dihydroergotamine, ergostine, butalbital, phenobarbital, acetaminophen, diclofenac sodium, ketoprofen, ketorolac, ibuprofen, prioxicam, naproxen, acetylsalicylic acid, flurbiprofen, tolfenamic acid, butorphanol, meperidine, methadone, sumatriptan, naratriptan, razatriptan, zolmitriptan, almotriptan, eletriptan, dexamethasone, hydrocortisone, isometheptene, chlorpromazine, diazepam, droperidol, valproic acid, gabapentin, topiramate, divalproex sodium, or an antinausea agent selected from the group consisting of metoclopramide, prochlorperazine, domperidone, ondansetron, tropisetron, dolasetron, nabilone, dronabinol, levonantradol, CP55,940, SR 144528, aprepitant, cyclizine, and promethazine, BIBN-4096BS, SB-(+)-273779, each alone or in combination with another pharmaceutical agent and/or with a pharmaceutically acceptable excipient suitable for intravaginal or transvaginal delivery of said agent to a human female subject, said composition consisting essentially of a combination of an effective amount of said antimigraine and/or antinausea drug with at least a mucoadhesive agent promoting adhesion of the composition to the vaginal mucosa for delivery of the drug to the vaginal mucosa or with a combination comprising at least a mucoadhesive agent, a permeation enhancer, and a lipophilic or hydrophilic carrier for transmucosal delivery of the agent through the vaginal mucosa to the general circulation.

Yet another aspect of this invention is an intravaginal composition for vaginal or transmucosal vaginal delivery of an antimigraine and/or antinausea drug, said composition administered directly or incorporated into a device selected from the group consisting of an intravaginal tampon, intravaginal ring, intravaginal pessary, intravaginal sponge, intravaginal tablet, intravaginal film, intravaginal foam, intravaginal xerogel, or intravaginal strip incorporated with a composition comprising an antimigraine and/or antinausea drug selected from the group consisting of ergot alkaloids and derivatives, antihistamines, barbiturates, non-steroidal anti-inflammatory agents, analgesics, serotonin antagonists, cannabinoids, calcitonin gene-related peptide (CGRP) antagonists, neurokinin-1 antagonists, steroids, sympathomimetics, tranquilizers, and antiepileptics each alone or in combination with an antinausea drug or further in combination with other pharmaceutical agents or pharmaceutically acceptable excipients, said composition formulated as a cream, lotion, foam, film, tablet, capsule, ointment, suppository, liposomal suspension, microemulsion, bioadhesive microparticles or microcapsules, bioadhesive nanoparticles or nanacapsules, solution, emulsion or gel.

Another aspect of this invention is an intravaginal device for vaginal or transmucosal vaginal delivery of an antimigraine and/or antinausea drug, said device selected from the group consisting of an intravaginal tampon, intravaginal ring, intravaginal pessary, intravaginal sponge, intravaginal tablet, intravaginal xerogel, or intravaginal strip incorporated with a composition comprising an antimigraine and/or antinausea drug consisting of ergot alkaloids and derivatives, antihistamines, barbiturates, non-steroidal anti-inflammatory agents, analgesics, serotonin antagonists, neurokinin-1 antagonists, cannabinoids, calcitonin gene-related peptide (CGRP) antagonists, steroids, sympathomimetics, tranquilizers and antiepileptics, formulated alone or in combination with other pharmaceutical agents or pharmaceutically acceptable excipients as a cream, lotion, foam, film, tablet, capsule, ointment, suppository, liposomal suspension, microemulsion, bioadhesive microparticles or microcapsules, bioadhesive nanoparticles or nanacapsules, solution, emulsion or gel, incorporated within said device.

Still yet another aspect of this invention is a medicated intravaginal device incorporated with a mucosal composition comprising in dosage unit form, an antimigraine and/or antinausea drug selected from the group consisting of but not limited to the group of ergotamine, dihydroergotamine, ergostine, butalbital, phenobarbital, acetaminophen, diclofenac sodium, ketoprofen, ketorolac, ibuprofen, prioxicam, naproxen, acetylsalicylic acid, flurbiprofen, tolfenamic acid, butorphanol, meperidine, methadone, sumatriptan, naratriptan, razatriptan, zolmitriptan, almotriptan, eletriptan, dexamethasone, hydrocortisone, isometheptene, chlorpromazine, diazepam, droperidol, valproic acid, gabapentin, topiramate, divalproex sodium, metoclopramide, prochlorperazine, domperidone, ondansetron, tropisetron, dolasetron, nabilone, dronabinol, levonantradol, CP55,940, SR 144528, aprepitant, cyclizine, and promethazine, BIBN-4096BS, SB-(+)-273779 alone or in combination and/or further in combination with other pharmacological agents or a pharmaceutically acceptable excipients suitable for intravaginal or transvaginal delivery of said agent to a human female, said composition consisting essentially of a combination of an effective amount of said antimigraine and/or antinausea drug with at least a mucoadhesive agent promoting adhesion of the composition to the vaginal mucosa for delivery of the drug to the vaginal mucosa or with at least a mucoadhesive agent, a permeation enhancer, and a lipophilic or hydrophilic carrier for transmucosal delivery of the agent through the vaginal mucosa to the systemic blood circulation.

### BRIEF DESCRIPTION OF THE DRAWINGS

FIG. 1 is a graph showing plasma concentration-time profiles of the antimigraine drug sumatriptan (−0.7 mg/kg) following vaginal administration to anesthetized female New Zealand rabbits. Radiactive drug concentrations were determined using liquid scintillation counting (n=3, average ±S.D.)

FIG. 2 shows results of comparison of intravenous and vaginal delivery of antinausea drug metoclopramide. FIG. 2A is a graph illustrating plasma concentration-time profiles of the antinausea drug metoclopramide following vaginal administration of the drug to the female New Zealand rabbits (˜0.5 mg/animal). Open circles (∘) represent intravenous dosing, closed squares (▪) represent vaginal administration. FIG. 2B is a graph showing comparison of dose-normalized plasma concentrations of metoclopramide in female New Zealand rabbits (˜0.5 mg/animal) following oral (Δ) and vaginal (▪) administration. Drug concentrations were determined by HPLC (n=3 and 4, resp., average ±S.D.)

FIG. 3 is a representation of the female reproductive organs. FIG. 3A is cross-sectional representation of a portion of the female reproductive organs including the systemic circulation and vagina in the upright orientation. FIG. 3B is a cross-sectional side view representation of a portion of the female reproductive organs including the uterus and vagina.

FIG. 4 shows placement of a vaginal device of a drug delivery system according to the present invention. FIG. 4A is a front view and FIG. 4B is a cross-sectional side view representation of the vaginal area adjacent the cervix showing placement of a tampon device incorporating an annular delivery composition.

FIG. 5 is the representation of FIG. 3B showing placement of a tampon device according to the present invention.

FIG. 6 is the representation of FIG. 3B showing placement of a tampon device incorporating a distal porous foam section.

FIG. 7 is the representation of FIG. 3B showing placement of a tampon device incorporating a distal porous foam cup. FIG. 7A is a cross-sectional view of the embodiment shown in FIG. 7, taken in the direction indicated by the arrows labeled 7A in FIG. 7.

FIG. 8 is an alternate arrangement to the one shown in FIG. 7 in which medication is contained in the entire porous foam cup.

FIG. 9 is the representation of FIG. 3B showing placement of a tampon device incorporating a distal suppository or gel capsule. FIG. 9A is a cross-sectional view of the embodiment shown in FIG. 9, taken in the direction indicated by the arrows labeled 9A in FIG. 9.

FIG. 10 is the representation of FIG. 3B showing placement of a tampon device incorporating a distal foam cup having “fingers.” FIG. 10A is a side view of the distal porous foam cup.

FIG. 11 is the representation of FIG. 3B showing placement of a tampon device incorporating a scoop-shaped distal porous foam section.

FIG. 12 is a side view of the embodiment shown in FIG. 11.

FIG. 13 is a front view of the embodiment shown in FIG. 11.

FIG. 14 is the representation of FIG. 3B showing placement of a tampon-like device incorporating distal fibers containing concentrated medication.

FIG. 15 is the representation of FIG. 3B showing placement of a tampon-like device incorporating non-absorbent tubing having a distal opening.

FIG. 16 is the tampon drug delivery system of FIG. 15 in a dehydrated and sheathed state.

FIG. 17 is the tampon drug delivery system of FIG. 16 showing deployment of the tampon.

### DEFINITIONS

As used herein:

Drug” or “agent” means a therapeutically effective compound suitable for treatment, management, or control of migraine or other pathophysiological condition accompanied by nausea and/or vomiting.

“Pharmacological agent” or “therapeutic agent” means an antimigraine drug, an antinausea drug, a mixture of both, or any other therapeutically acceptable and effective agent.

“Antimigraine drug” means an agent involved in treatment of disease, typically migraine condition, by means of chemical substance or drug that reduces pathophysiological symptoms associated with the disease.

“Antinausea drug” or “antiemetic drug” means a chemical compound, which is suitable to partially or completely suppress nausea and/or vomiting typically associated with the migraine condition or any other disease state or condition, such as chemotherapy, radiotherapy, pregnancy, surgery, or administration of drugs which cause nausea.

“Pharmaceutical ingredient” or “excipient” means a pharmacologically inactive pharmaceutically acceptable compound required to prepare drug delivery systems and/or devices with desired properties.

“Rapid delivery” means initial immediate release and delivery of the drug, followed by a time-dependent reduction in release and delivery from the formulation or device.

“Continuous delivery” means continuous and uninterrupted release of the drug from the formulation or device and delivering such drug in continuous manner.

“Pulsed delivery” means a release and delivery of the drug in intermittent intervals. Such pulsed delivery may be provided, for example, by formulating the drug in individual layers interspaced with inactive layers of dissolvable coatings or by using different pharmaceutical ingredients.

“Chemotherapy” means a treatment of cancer using a chemical agent involved in treatment of disease by means of chemical substance or drug that exhibits cytostatic and/or cytotoxic effects on tumor cells.

“Interesterified stone oil” means a vegetable oil ethoxylated by replacing part of glycerol of the glycerides contained in vegetable oil by polyoxyethylene glycols of different lengths. Such replacement results in hydrophilic properties. Example of the interesterified stone oil is LABRAFIL®, particularly LABRAFIL® M1944 CS, commercially available from Gattefossé.

“Mucosa” or “mucosal tissue” means surface epithelial tissue that is accessible from the outside of the body without surgical procedures.

“Mucosal” or “mucoadhesive” means a composition which is suitable for administration to the mucosal tissue and adhere to such mucosal tissue.

“Permeation enhancer” means a compound that promotes transfer of an agent across a mucosal barrier, which is increasing the mass transfer into as well as through the epithelium.

### DETAILED DESCRIPTION OF THE INVENTION

The current invention concerns a method, composition, and a device for topical mucosal or transmucosal vaginal delivery of antimigraine and/or antinausea drugs for treatment and control of migraine and headache pain, nausea and vomiting associated with migraine or with other diseases or conditions, such as, for example, following administration of chemotherapeutic drugs, radiotherapy, surgery, or accompanying menstrual period, pregnancy, breastfeeding, and menopause. The beneficial therapeutic effect according to this invention is achieved by contacting vaginal mucosa with a mucosal composition or with a device incorporated with a mucosal composition comprising an antimigraine or an antinausea agent or a combination thereof or a combination of each of these agents alone or their combination with other therapeutically effective agents.

Transvaginal delivery systems according to the invention offer a viable alternative to deliver antimigraine and/or antiemetic (antinausea) drugs to a female subject suffering from migraine or other disease or conditions that result in headache, nausea, and vomiting. In contrast to the oral route, administration of antimigraine and/or antinausea drugs using vaginal drug delivery systems does not stimulate vomiting reflexes and, therefore, reduce migraine-associated vomiting. In addition, drugs delivered transmucosally through vaginal mucosa enter the systemic circulations system bypassing the first pass liver detoxification and degradation. Consequently, this route of administration is particularly advantageous for drugs, including antimigraine and antinausea drugs, that undergo substantial hepatic first-pass metabolism.

This type of drug delivery is particularly suitable for treatment of migraine because, typically, migraine is accompanied by severe headaches, nausea and vomiting. Nausea and vomiting prevent effective treatment of migraine by oral route because the recipient is usually not able to hold the drug in the stomach for long enough time to achieve needed relief from the pain and nausea. Thus, the oral drug delivery for treatment of migraine and other conditions accompanied by nausea and vomiting is unpredictable and ineffective insofar as the actual delivered drug dose. Furthermore, women are generally accustomed, on a routine basis, to the insertion of a vaginal device such as a tampon for menstrual control and are expected to embrace this alternative route of delivery for therapeutic control of migraine condition without dramatic emotional distress.

The method of the invention provides a novel route of delivery of antimigraine and/or antinausea drugs for treatment and control of the migraine condition as well as other situations, such as drug-induced nausea, chemotherapy, radiotherapy, post-surgery nausea, pre-operative medication, PMS, menstruation, pregnancy, breastfeeding and menopause, where administration of these drugs provides relief of similar symptoms in the female subject. The method avoids drug administration into the gastrointestinal tract of nauseated patients, protects the therapeutic agent from extensive hepatic first-pass metabolism, permits rapid, continuous or pulsed delivery of the antimigraine and/or antinausea drugs, and achieves therapeutically effective concentrations of such agents in the blood circulation.

The method for treatment and control of headache pain, nausea and vomiting comprises administration of a mucoadhesive composition containing a therapeutically effective amount of the appropriate antimigraine or antinausea agent alone or incorporated into a vaginal device of the invention before or after onset of migraine, before surgery, during menstrual period or pregnancy, or before or after headache, nausea and vomiting appear. The composition or the device are introduced intravaginally to the mucosa for direct topical effect or, preferably, for absorption and transport through the mucosa and transmucosally to the systemic circulation.

A device, composition, and a method for administration of antimigraine and/or antinausea drugs are suitable for both the transmucosal delivery to the systemic circulation and a topical delivery to the vaginal mucosa.

Administration of antimigraine and/or antinausea drug via the vaginal route also reduces the portion of the drug which would be eliminated by the liver during its first pass circulation in the blood system, which further enhances their therapeutic effect.

I. Mucosal and Transmucosal Vaginal Delivery of Antimigraine or Antinausea Drugs

A method of transmucosal and topical mucosal vaginal delivery comprises intravaginal administration of the mucoadhesive vaginal composition or the intravaginal device of the invention incorporated with such composition. The composition or the device delivers an antimigraine and/or antinausea to and through the vaginal mucosa into the systemic blood circulation. Such delivery occurs without oral administration and thus eliminates therapy-induced vomiting typically occurring with oral administration of antimigraine and/or antinausea drugs for treatment and control of headache pain, nausea and vomiting, which are primary symptoms associated with migraine but are also associated with other conditions or diseases and occur, for example, as a result of administration of chemotherapeutic drugs, following radiotherapy, before or after surgery, during menstrual period, pregnancy, breast-feeding, and menopause, among others.

A. Advantages of Vaginal Delivery

Existing therapeutic approaches used to control migraine symptoms and/or headache pain, nausea and vomiting mostly depend on oral, intravenous, nasal or rectal drug delivery systems. Unfortunately, drug administration via the gastrointestinal tract in migraine patients stimulates rather than eliminates vomiting and, consequently, results in inadequate treatment of these conditions. Alternatively, parenteral intramuscular or subcutaneous injections, nasal sprays, or insertion of rectal suppositories are employed to bypass problems and difficulties encountered with oral administration of these drugs in migraine patients. In this regard, injection methods usually require visit to a medical facility and assistance of a trained health care professional, whereas many patients find insertion of a rectal dosage form uncomfortable and/or emotionally unpleasant. Nasal delivery systems for migraine therapy have been only partially successful as the amount needed to achieve a relief from pain and nausea needs to consider first pass liver deactivation of the substantial amount of the drug and thus is efficacious only for drugs that are highly resistant to hepatic metabolism.

The vaginal route of delivery allows rapid, continuous or pulsed delivery of drugs in a patient-controlled environment without the need to have access to a skilled health care professional in a doctor's office or hospital. Using the mucosal composition and intravaginal device of the invention, an effective dose of a desired therapeutic agent can be delivered reproducibly to the systemic circulation while vomiting, which frequently occurs after oral drug administration in migraine patients is prevented, and eliminates parenteral injection with all its adverse effects and requirements. Furthermore, since the blood circulation into which the drugs are delivered through vaginal mucosa circumvents the liver first-pass circulation, the portion of the vaginally delivered drug is substantially increased compared to the portion of the drug administered orally.

The invention, thus, concerns discovery of an improved delivery route of antimigraine and antinausea drugs that overcomes the side effects and limitations observed during oral, parenteral, and nasal administration of these agents in subjects suffering from headaches or nausea. The invention utilized anatomic advantage of female subjects by focusing the delivery of drug therapy directly to the vaginal mucosa using a specifically formulated mucoadhesive composition or an intravaginal device incorporated with such specifically formulated composition containing a therapeutically effective amount of an appropriate antimigraine and/or antinausea agent. Such composition promotes adhesion of the composition, including the drug released from the device, to the vaginal mucosa and further promotes a transmucosal delivery of the drug across the vaginal epithelial barrier into the systemic blood circulation.

The therapy according to the invention is suitable for treatment and control of headache pain, nausea, and vomiting associated with migraine, as a result of administration of chemotherapeutic drugs, before or after surgery, during menstrual period, pregnancy, breast-feeding, radiation, and menopause.

Contacting the vaginal mucosa with antimigraine and/or antinausea incorporated into the composition according to this invention for topical and transmucosal delivery greatly increases therapeutically effective concentrations in the blood systems and circumvents the gastrointestinal tract, parenteral injections, and first-pass hepatic elimination.

The newly developed vaginal delivery strategy of antimigraine and/or antinausea drugs according to the invention, therefore, represents an important improvement in the systemic delivery of these drugs and an important advancement of migraine therapy as well as the therapy of conditions leading to headache pain, nausea and vomiting associated with other diseases and conditions as described above.

B. Confirmation of Transmucosal Delivery

Mucosal or preferably transmucosal delivery according to the invention is suitable for delivery of antimigraine as well as antinausea drugs. Examples of such therapeutic agents are ergot alkaloids and derivatives, antihistamines, barbiturates, non-steroidal anti-inflammatory agents, analgesics, serotonin antagonists, neurokinin-1 antagonists, cannabinoids, calcitonin gene-related peptide (CGRP) antagonists, steroids, sympathomimetics, tranquilizers, and antiepileptics.

While the mechanisms involved in the pathogenesis of migraine are still not completely understood, it is hypothesized that migraine is a disorder blood vessels in the brain (Ann. Neurol., 16: 157-168 (1984); Radiol. Technol., 74:281-314 (2003)). Alterations in the activity of 5-hydroxytryptamine-containing neurons in the raphe nuclei and/or norepinephrine-containing pathways lead to depolarization of trigeminoperivascular nerves and release of vasoactive neuropeptides. This results in vasodilatation of brain arteries and exacerbation of pain, and, ultimately, lead to neurogenic inflammation. Transmissions of pain impulses to the central nervous system are further responsible for associated symptoms such as nausea and vomiting that typically accompany the migraine condition. Antimigraine drugs are, therefore, valuable agents to provide relief from these multiples symptoms occurring during a migraine condition.

In addition to the therapeutic benefit of antinausea drugs in migraine therapy, effective systemic delivery of these agents across the vaginal mucosa as described in this invention also offers a dramatic advantage in the treatment and control of nausea, and vomiting that occur as a result of administration of chemotherapeutic drugs, before or after surgery, during menstrual period, pregnancy, breast-feeding, radiation and menopause.

The transmucosal delivery of antimigraine and antinausea agents across the vaginal mucosa results is a viable alternative to oral delivery. Such delivery was confirmed in in vivo pharmacokinetic studies performed using a rabbit model. As discussed above, oral administration of antimigraine and/or antinausea drugs will stimulate vomiting rather then eliminate nausea and, consequently, leads to uncontrollable reduction of the drug absorption. Therefore, transvaginal delivery systems of the invention, as experimentally confirmed, proves to be an effective means of delivering therapeutic quantities of antimigraine and antinausea drugs in the female subjects.

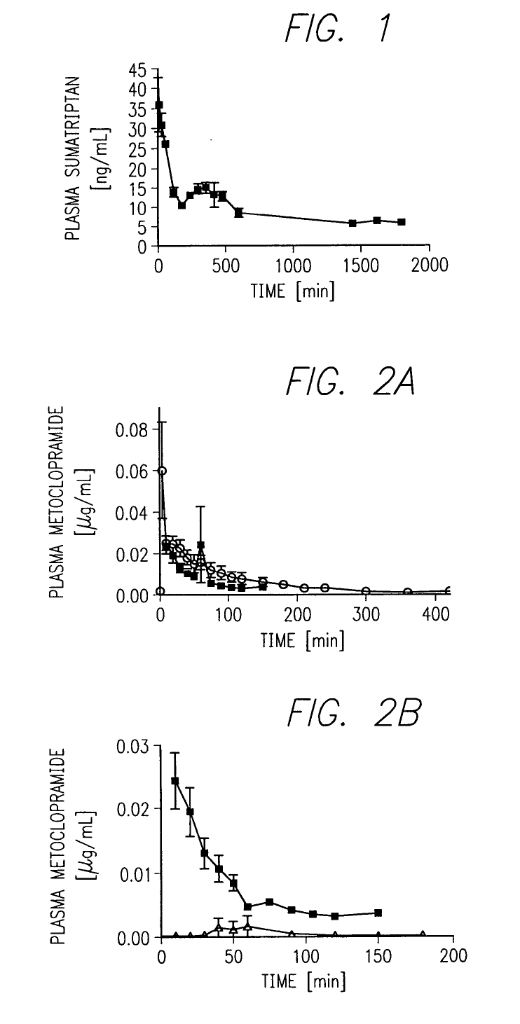

The objective of these studies was to determine the rate and extent to which the antimigraine drug sumatriptan and the antinausea agent metoclopramide reach the systemic circulation following vaginal administration. Furthermore, studies were designed to compare the absolute bioavailabilities of these agents when administered orally and vaginally. To achieve this objective, the pharmacokinetic profiles of these drugs in plasma were compared after intravenous, oral, and vaginal administration. Results are seen in FIGS. 1 and 2.

Plasma pharmacokinetic profiles of sumatriptan and metoclopramide were determined in anesthetized female white New Zealand rabbits after intravenous, vaginal, and oral administration. For sumatriptan, a dose of 0.7 mg/kg body weight was used, supplemented with a trace amount of [³H]sumatriptan for analytical purpose. Plasma concentration—time profiles were analyzed using the non-compartmental module of WinNonlin. Results are shown in FIGS. 1 and 2 and in Table 1.

FIG. 1 shows plasma concentration-time profiles of the antimigraine drug sumatriptan following vaginal administration in anesthetized female New Zealand rabbits (˜0.7 mg/kg). Radioactive drug concentrations were determined using liquid scintillation counting (n=3, average±S.D.).

FIG. 1 demonstrates that vaginal administration of a suppository comprising sumatriptan, water, the mucoadhesive HPMC, the permeation enhancer TRANSCUTOL®, and SUPPOCIRE® as the lipophilic carrier, results in significant plasma concentrations of this antimigraine drug using the rabbit in vivo model. Similar to the profile following intravenous administration, vaginal delivery achieves peak drug levels within a few minutes. This implies rapid onset of the pharmacological activity, i.e., resulting in fast relief of headache pain in migraine patients.

Furthermore, vaginal administration of sumatriptan in the rabbit model leads to the unique feature of a second peak in the plasma without administration of a consecutive dose. This is an example of pulsed drug delivery that provides the unique therapeutic advantage to suppress subsequent symptoms of the migraine condition without administration of a second dose.

As seen in FIG. 1, pharmacokinetic analysis of the sumatriptan plasma profile following intravenous, vaginal, and oral administration revealed a significantly increased terminal half-life after vaginal and oral dosing seen in Table 1.

TABLE 1
Pharmacokinetic Parameters of Sumatriptan in New Zealand Rabbits
Following Intravenous, Vaginal, and Peroral Administration

| Parameter | intravenous | vaginal | peroral |
|---|---|---|---|
| Dose [mg × kg⁻¹] | 0.74 − 0.77 | 0.64 − 0.70 | 0.80 − 0.85 |
| cₘₐₓ₁[ng × mL⁻¹] | 15360 ± 4527 | 36 ± 12 | 894 − 512 |
| tₘₐₓ₁[min] | 1 | 15 | 100 |
| Cₘₐₓ₂[ng × mL⁻¹] | N/A | 15 ± 2 | N/A |
| tₘₐₓ₂[hr] | N/A | 300 | N/A |
| AUC [μg × min × mL⁻¹] | 10868 ± 90 | 23 ± 0.1 | 958 ± 962 |
| t_{1/2}[min] | 191 ± 6 | 1369 ± 300 | 1210 ± 163 |

Pharmacokinetic parameters were calculated from plasma drug concentrations using WinNonlin.

This so-called “flip-flop” phenomenon is the result of a change in the rate-limiting step of sumatriptan pharmacokinetics when drug is administered via an extravascular route. Generally, it is concluded that transfer across the mucosal barrier is responsible for this change in rate-limiting step. However, it is also possible that the release of sumatriptan from the vaginal suppository is kinetically the slowest step controlling absorption. Addition of the mucoadhesive excipient HPMC (1.5% (w/w)) is designed to maintain close contact between the drug and the vaginal epithelium. Simultaneously, HPMC is known to delay drug release from drug delivery systems due to formation of a highly viscous gel in the presence of water.

The antinausea drug metoclopramide was investigated in the above described rabbit model using intravenous, vaginal, and oral administration of 0.2 mg drug per animal. Blood samples were removed at various time periods up to 6 hours, and plasma concentrations of metoclopramide were quantified using a HPLC method. Model-independent pharmacokinetic analysis was performed using WinNonlin.

For the study of antinausea drug, blood samples were removed at various time periods up to 7 hours, and plasma concentrations of metoclopramide were quantified using a HPLC method described in Int. J. Clin. Pharmacol., Ther., 40:169-174 (2002). Results are shown in FIGS. 2A and 2B and in Table 2.

FIG. 2A shows plasma concentration-time profiles of the antinausea drug metoclopramide following intravenous and vaginal administration in anesthetized female New Zealand rabbits (˜0.5 mg/animal). Drug concentrations were determined using a modified HPLC method. Open circles represent intravenous dosing, closed squares vaginal administration.

FIG. 2B shows comparison of dose-normalized plasma concentrations of metoclopramide in female New Zealand rabbits (˜0.5 mg/animal) following oral (Δ) and vaginal (▪) administration. Drug concentrations were determined by HPLC (n=3-4, average ±S.D.).

Similar to an intravenous injection of this antinausea drug, vaginal administration of this agent results in high plasma levels within 10 min as also shown in Table 1. As confirmed by these results, the pharmacological effect of this drug in suppressing nausea and vomiting is almost instantaneously achieved following administration. These results are highly desirable for the use of antinausea drugs that are considered the drugs of first line emergency.

When compared to the plasma profile measured after oral administration (FIG. 2B), which is not desired in patients suffering from nausea because vomiting reflex can be stimulated as a result of liquid ingestion and/or increased pressure in the stomach, these studies clearly demonstrate the advantage of vaginal delivery over standard oral administration practice of antinausea drugs. Not only are the maximum plasma concentrations significantly lower after oral administration than after vaginal dosing, but also the time required to reach this maximum drug peak in the plasma is dramatically increased when drug is administered orally.

It is, therefore, evident that therapeutic effects in nauseated patients are more likely to occur faster after vaginal administration than after oral dosing. Although the physicochemical properties of this antinausea drug are not the most favorable for rapid transfer across the vaginal mucosa, the presence of the potent permeation enhancer TRANSCUTOL® facilitates transepithelial transport of metoclopramide across this biological barrier.

Table 2 compares the pharmacokinetic parameters determined for metoclopramide in the rabbit model following intravenous, vaginal, and oral administration.

TABLE 2
Pharmacokinetic Parameters of Metoclopramide in New Zealand
Rabbits Following Intravenous, Vaginal, and Peroral Administration

| Parameter | intravenous | vaginal | peroral |
|---|---|---|---|
| Dose [mg × kg⁻¹] | 0.11 − 0.12 | 0.11 − 0.12 | 0.11 |
| Cₘₐₓ₁[ng × mL⁻¹] | 61.8 ± 45.3 | 24.4 ± 8.9 | 2.3 ± 4.0 |
| tₘₐₓ₁[min] | 5 | 10 | 60 |
| AUG [ng × min × mL⁻¹] | 3845 ± 1415 | 1268 ± 252 | 180 ± 265 |
| t_{1/2}[min] | 134 ± 114 | 49 ± 24 | 149 ± 169 |

Pharmacokinetic parameters were calculated from plasma drug concentrations using WinNonlin.

The maximum plasma concentration (cₘₐₓ) measured after drug administration via the various routes clearly favors the intravenous route for highly critical situations. Nevertheless, in any other condition in the female where immediate relief from nausea/vomiting is desired, vaginal administration appears more beneficial because it achieves higher plasma levels within a shorter time period than after oral dosing. Overall, oral and vaginal absorptions are incomplete. The greater AUC∞, which corresponds to the extent of drug reaching the systemic circulation, following vaginal administration could lead to enhanced therapeutic effects, less frequent dosing, and greater patient compliance. In addition, statistical analysis of these parameters implies a more reproducible delivery of metoclopramide from the vaginal device than administered as an oral solution.

II. Method for Delivery of the Antimigraine/Antinausea Therapy

A method of the invention is developed for and is particularly suitable for bypassing extensive hepatic first-pass elimination of antimigraine and/or antinausea drugs and for reducing the symptoms of nausea and vomiting.

The method, useful for treatment, management, and control of headache pain, nausea, and vomiting, which are primary symptoms associated with migraine but are not only limited to this condition but can also occur as a result of other conditions such as administration of chemotherapeutic drugs or surgery as described in detail above, comprises steps of contacting the vaginal epithelium lining the vaginal cavity with a mucoadhesive composition or with an intravaginal device incorporated with such composition. Said method comprises at least one antimigraine drug or one antinausea agent selected from the group consisting of ergot alkaloids and derivatives, antihistamines, barbiturates, non-steroidal anti-inflammatory agents, analgesics, serotonin antagonists, neurokinin-1 antagonists, cannabinoids, calcitonin gene-related peptide (CGRP) antagonists, steroids, sympathomimetics, tranquilizers, and antiepileptics, alone or in combination with another antimigraine drug or said antinausea agent, or in combination with another pharmaceutical agent or a pharmaceutically acceptable excipient, and maintaining said composition or device in contact with said vaginal mucosa for a period of time permitting a rapid, continuous or pulsed delivery of the agent to or through the vaginal mucosa and necessary to deliver a therapeutic dose of the antimigraine and/or antinausea drug. Such time is typically from several minutes to several hours.

The delivery route utilizes the transmucosal composition directly or incorporated into an intravaginal device for transmucosal delivery and comprises delivery of a combination of the antimigraine and/or antinausea agent with a mucoadhesive agent, carriers and, optionally, permeation enhancing agents and solublizing excipients for transvaginal delivery.

Additionally, more than one drug may be present in the composition of an antimigraine or antinausea composition or any other pharmacologically active agent suitable for the treatment, management, and control of headache pain, nausea, and vomiting may be added. All combinations and variations are intended to be within the scope of the invention.

Hence, the current method includes the delivery of antimigraine or antinausea drugs in a combination with drugs that may reduce inflammation, have analgesic effects, modulate cerebral blood flow, and such other therapeutically active agents.

Specifically, the antimigraine compounds suitable for delivery according to the method are selected from but are not limited to the group consisting of ergotamine, dihydroergotamine, ergostine, butalbital, phenobarbital, acetaminophen, diclofenac sodium, ketoprofen, ketorolac, ibuprofen, prioxicam, naproxen, acetylsalicylic acid, flurbiprofen, tolfenamic acid, butorphanol, meperidine, methadone, sumatriptan, naratriptan, razatriptan, zolmitriptan, almotriptan, eletriptan, dexamethasone, hydrocortisone, isometheptene, chlorpromazine, diazepam, droperidol, valproic acid, gabapentin, topiramate, divalproex sodium, metoclopramide, prochlorperazine, domperidone, ondansetron, tropisetron, dolasetron, nabilone, dronabinol, levonantradol, CP55,940, SR 144528, aprepitant, cyclizine, and promethazine, BIBN-4096BS, SB-(+)-273779. These compounds may be administered alone or in combination with other pharmacological agents or a pharmaceutically acceptable excipient.

The method may be practiced either by administering the mucoadhesive composition directly to the vagina as a solution, gel, cream, lotion, ointment, foam, film, suppository, liposomal suspension, microemulsion, capsule, tablet, microparticles, microcapsules, nanoparticles, nanocapsules, or by inserting the intravaginal device incorporated with the above described mucoadhesive composition comprising an antimigraine and/or antinausea drug. The device suitable for these purposes is selected from the group consisting of an intravaginal tampon, intravaginal ring, intravaginal pessary, intravaginal sponge, intravaginal tablet, intravaginal capsule, intravaginal patch, intravaginal iontophoretic system, intravaginal cup, and intravaginal strip.

The direct contact permits an immediate rapid, extended continuous, or intermittent, pulsed delivery that leads to efficacious treatment. Such direct-treatment also permits use of only such a dosage of the antimigraine and/or antinausea agent as it is therapeutically required for topical and/or systemic treatment of the affected organ.

The method of the invention, suitable for treatment, management, and control of headache pain, nausea, and vomiting depends on a specifically formulated mucosal composition comprising the antimigraine and/or antinausea drug or an intravaginal device incorporated with said composition and/or inserting said composition or device into the vaginal cavity for a period of time that is required to achieve a therapeutic effect of said mucosal composition. The composition is formulated to deliver the antimigraine and/or antinausea agent to the target organ for treatment, management, and control of headache pain, nausea, and vomiting. For each of the treatments, the drug is formulated differently.

The method for treatment, management, and control of headache pain, nausea, and vomiting using transmucosal delivery of the drug to the systemic blood circulation involves adding to the composition of the invention components promoting transfer of an agent across the vaginal epithelial barrier. Such components are added in amounts sufficient to facilitate transmucosal delivery to the cardiovascular system.

Transmucosal treatment is based on the concept that the upper vagina and the uterus have specific blood flow characteristics, either by a portal type circulation or by venous and lymphatic channels, permitting preferential transport and delivery of pharmacological agents from the vaginal mucosa directly to the systemic blood circulation, thereby bypassing the gastrointestinal tract and liver, where is most of antimigraine and/or antinausea drugs experience a substantial elimination through metabolism.

The most specific demonstration of the transvaginal concept has been achieved by inventors with several types of drugs, as described in U.S. Pat. Nos. 6,086,909, and 6,197,327, 6,461,779 B1, and 6,572,874 incorporated by reference. Antimigraine agents and/or antinausea drugs, when properly formulated, are transported through the vaginal wall in the same manner as described in the above patents.

The composition is a stand-alone treatment or it is incorporated into a suitable intravaginal delivery device, which assures close contact with the vaginal mucosa.

The composition or the medicated device according to the method is administered or applied, that is, inserted intravaginally for a specified time frame from about ten minutes to several hours, preferably half an hour, once, twice, or several times per day or week as needed or according to an optimized treatment regimen. The device is typically provided in dry or wet form or may be wetted prior to insertion.

The device of the invention provides a continuous drug depot, which allows continuous and uninterrupted delivery of drug to the vaginal mucosa over a long period of time. Fourth, and very important, is the reduction in side effects due to avoidance of repeated parenteral injections of antimigraine and/or antinausea drugs.

III. Mucoadhesive Compositions

A mucoadhesive composition of the invention for transmucosal delivery of antimigraine or antinausea drugs consists typically of four essential components. These components are a pharmacologically active agent that achieves a therapeutically desired effect, a mucoadhesive agent that provides close contact of the composition with the vaginal epithelium, a lipophilic or hydrophilic carrier that assures safe patient handling and enhances surface exposure of the drug to the vaginal mucosa, and a permeation enhancer that facilitates transfer of the pharmacologically active agents across the epithelial barrier into submucosal tissue and the systemic blood circulation.

For topical delivery to the vaginal mucosa, the composition consists of at least three components, a pharmacologically active agent, a mucbadhesive agent, and a lipophilic or hydrophilic carrier. The pharmacologically active agent is either an antimigraine and/or antinausea drug. These agents are formulated either alone or in admixture with another pharmacological agent or a pharmaceutically acceptable excipient. All the above mentioned components of the composition must be suitable for administration to the vagina or for incorporation into an intravaginal device for the vaginal or transmucosal vaginal delivery of the drug through the vaginal mucosa into the general blood circulation. The therapeutically active compound is present in a dose sufficient to assert its therapeutic effect, typically from about 0.001 to about 3000 mg, preferably from 0.1 to 1000 mg, most preferably from 10 to 500 mg.

The mucoadhesive composition is typically formulated in therapeutic unit dosage forms and contains an antimigraine or an antinausea drug selected from ergot alkaloids and derivatives, antihistamines, barbiturates, non-steroidal anti-inflammatory agents, analgesics, serotonin antagonists, neurokinin-1 antagonists, cannabinoids, calcitonin gene-related peptide (CGRP) antagonists, steroids, sympathomimetics, tranquilizers, and antiepileptics, alone, in combination, or in combination with other pharmacological agents or pharmaceutically acceptable excipients for intravaginal or transvaginal delivery to a human female subject.

The composition typically contains from 0.001 to about 3000 mg, preferably from 0.1 to 1000 mg, of an antimigraine and/or antinausea drug, from about 0.1 to about 25% of mucoadhesive agent promoting adhesion of the composition to the vaginal mucosa, from about 5 to about 30% of a permeation enhancer assuring transfer of the drug across the vaginal epithelium, and from about 40 to about 95% of a lipophilic or hydrophilic carrier serving as a vehicle for the drug and, optionally, from about 0 to about 30%, preferably about 1 to 5%, of a solubilizing agent for increased transport of released pharmacological agent into the systemic blood circulation.

Other pharmaceutically acceptable excipients suitable for vaginal delivery, such as buffers, fillers, stabilizers, emulsifiers, and any such other excipient as is known in the art to be useful for these purposes may also be added.

Any component and/or excipient used in formulations of this invention needs to be approved for human use and acceptable for use in the vagina with understanding that not all excipients approved for oral use may be approved and/or suitable for vaginal use.

The composition is formulated as a solution, gel, cream, lotion, ointment, foam, film, suppository, liposomal suspension, microemulsion, capsule, tablet, microparticles, microcapsules, nanoparticles, or nanocapsules, and can be delivered as stand alone or incorporated within an intravaginal device.

The composition formulated as above can be incorporated into an intravaginal device or used as a coating for such device, for example, a tampon or tampon-like device medicated or coated with the above described mucoadhesive composition. Alternatively, the composition may be incorporated into a sponge, foam, film, tablet, capsule, ring, mucoadhesive patch, iontophoretic system, strip, pessary, or other material. Absorbent material or matrix of such device may be impregnated with a drug-containing solution, suspension, lotion, cream, emulsion, microemulsion, liposomes, microparticles, microcapsules, nanoparticles, or nanocapsules. The devices of the invention are described in greater detail below in Section IV.

Any form of drug delivery system that will effectively deliver the antimigraine and/or antinausea agent to the vaginal mucosa or transmucosally across the vaginal epithelium into the general blood circulation is intended to be included within the scope of this invention.

A. Components of the Mucoadhesive Composition

Individual components of the mucoadhesive composition are the antimigraine or antinausea drug, a mucoadhesive agent, a lipophilic or hydrophilic carrier and penetration enhancer or sorption promoter.

1. Antimigraine and Antinausea Agents

The antimigraine therapeutic agents are pharmacologically active compounds effective in the treatment, management, and control of headache pain and are generally selected from the following groups and types of compounds: ergot alkaloids and derivatives, barbiturates, non-steroidal anti-inflammatory agents, analgesics, serotonin antagonists, neurokinin-1 antagonists, calcitonin gene-related peptide (CGRP) antagonists, steroids, sympathomimetics, tranquilizers, and antiepileptics.

Specific antimigraine agents are selected from but not limited to the group of compounds, including ergotamine, dihydroergotamine, ergostine, butalbital, phenobarbital, acetaminophen, diclofenac sodium, ketoprofen, ketorolac, ibuprofen, prioxicam, naproxen, acethylsalicyclic acid, flurbiprofen, tolfenamic acid, butorphanol, meperidine, methadone, sumatriptan, naratriptan, razatriptan, zolmitriptan, almotriptan, eletriptan, dexamethasone, hydrocortisone, isometheptene, chlorpromazine, diazepam, droperidol, valproic acid, gabapentin, topiramate, divalproex sodium, and any other compound known now or which will become known in future to have similar antimigraine properties. All these compounds are intended to be covered by this invention.

Antinausea drugs are generally compounds which suppress nausea and/or prevent vomiting. Pharmacological agents with antimigraine effect are selected from but not limited to the group of drugs represented by metoclopramide, prochlorperazine, domperidone, ondansetron, tropisetron, dolasetron, nabilone, dronabinol, levonantradol, CP55,940, SR 144528, aprepitant, cyclizine, and promethazine, BIBN-4096BS, SB-(+)-273779. Any other compound known now or which will become known in future to have similar antinausea properties is intended to be covered by this invention.

The compounds of the invention are administered in doses ranging from about 0.001 to about 3000 mg, preferably from about 0.1 to 1000 mg, and most preferably from about 10 to 500 mg. Exemplary dosages for ergotamine, for example, are in the range from about 15 to about 300 mg/day, diclofenac sodium from about 100 to about 500 mg/day, sumatriptan from about 20 to 500 mg/day, zolmitriptan from about 10 to about 420 mg/day, naratriptan about 10 to about 350 mg/day, metoclopramide from about 20 to 120 mg/dose, prochlorperazine from about 25 to 150 mg/dose, ondansetron from about 30 to 210 mg/dose, dronabinol from about 10 to 50 mg/day, and promethazine from about 12 to about 80 mg/dose.

The antimigraine or antinausea drugs are formulated in said composition alone, in admixture of two or more or in admixture of the antimigraine agent and a antinausea drug, and/or in combination with another pharmacological agent or an acceptable pharmaceutical excipients.

2. Mucoadhesive Agents

For vaginal transmucosal delivery, the composition comprises, as an essential component, a mucoadhesive agent. The mucoadhesive agent permits a close and extended contact of the composition, or the drug released from said composition, with mucosal surface by promoting adherence of said composition or drug to the mucosa. The mucoadhesive agent is preferably a polymeric compound, such as preferably, a cellulose derivative but it may be also a natural gum, alginate, pectin, or such similar polymer. The most preferred cellulose derivative is hydroxypropyl methylcellulose available under the trade name METHOCEL®, commercially available from Dow Chemical Co.

The mucoadhesive agent is present in from about 0.1 to about 25%, by weight, preferably in from about 1.5 to about 15% and most preferably about 1.5-5%.

3. Sorption Promoters

The mucoadhesive composition additionally includes a sorption promoter present in from about 2 to about 30%, by weight. Sorption promoter assures a permeation and penetration of the drug, that is moving it through the tissue and entering systemic blood circulation through the vaginal mucosa.

Sorption promoters include non-ionizable glycol ester derivatives, such as polyethylene glycol caprylic/capric glycerides known as LABRASOL®, commercially available from Gattefossé, glycol derivatives with glycerol esters, such as oleic acid esters of propylene glycol and glycerol known as ARLACEL® 186, commercially available from Imperial Chemical Industries. Particularly preferred are non-ionizable glycol ether derivatives, such as, most preferably, ethoxydiglycol, known under its trade name TRANSCUTOL® and commercially available from Gattefosse, or interesterified stone oil, for example LABRAFIL M 1944CS, commercially available from Gattefosse. The interesterified stone oil is a vegetable oil ethoxylated by replacing part of glycerol of the glycerides contained in vegetable oil by polyoxyethylene-glycols.

4. Lipophilic and Hydrophilic Carriers

Depending on the drug affinity, the composition of the invention additionally comprises either the lipophilic or the hydrophilic carrier that is appropriate for the used antimigraine or antinausea agent. Such carrier is typically present from about 30 to about 95%, by weight.

The carrier is selected from such compounds for which the drug has low affinity. Thus the lipophilic carrier is appropriate and selected for formulation of the hydrophilic antimigraine or antinausea drug and the hydrophilic carrier is appropriate for formulation of the lipophilic antimigraine or antinausea drug.

i. Lipophilic Carriers

Preferred lipophilic carriers for use with hydrophilic drugs include any medium chain triglycerides and/or a saturated mono-, di- or triglyceride of fatty acids, particularly those having carbon chain of from 8 to 18 carbons, or a mixture thereof. Examples of the lipophilic carrier are saturated glycerides known and available under the trade name SUPPOCIRE® AS2 or CS2, and related compounds commercially available, for example, from Gattefosse, Westwood, N.J.

ii. Hydrophilic Carriers

Preferred hydrophilic carriers include polyethylene glycols of molecular weight between about 200 and 8000, OR derivatives or mixtures thereof, such as PEG 6000/PEG 1500, or PEG 6000/PEG 1500/PEG 400, or PEG 6000/PEG 400, or PEG 8000/PEG 1500, commercially available from, for example, Sigma/Aldrich, St. Louis, Mo.

5. Penetration Enhancers

Composition of the invention may additionally contain penetration enhancers, compounds which assist in improving penetration properties of the drug or their mixtures by changing the surface properties of the drugs or their mixtures, or drug containing solutions or suspensions. These compounds thus, in a way, act as solubilizers. Examples of the penetration enhancers are non-ionic surfactants.

The penetration enhancer may be added from about 1% to about 30%, as required.

6. Solubilizing Agents

The composition optionally includes also a solubilizing agent, such as complex-forming solubilizer citric acid, ethylenediamine-tetraacetate, sodium meta-phosphate, succinic acid, urea, cyclodextrin, polyvinylpyrrolidone, diethylammonium-ortho-benzoate, or micell-forming solubilizers such as tweens and spans, for example Tween 80. Other solubilizer useful for the compositions of this invention are polyoxyethylene sorbitan fatty acid ester, polyoxyethylene n-alkyl ethers, n-alkyl amine n-oxides, poloxamers, organic solvents, phospholipids and cyclodextrines.

The solubilizing agents may be added from about 0.1% to about 30%.

7. Additional Excipients

The composition of the invention may additionally contain other excipients, such as, fillers, emulsifiers, stabilizers, buffers, and others, as appropriate. Examples of these excipients are isostearylstearate, isopropyl myristate, glycerin, mineral oil, polycarbophil, carbomer 934P or 940, hydrogenated palm oil, glyceride, sodium hydroxide, sorbic acid, and purified water.

B. Preferred Formulations

All formulations which contains components of the invention in ranges given above are intended to be within the scope of this invention. Few compositions presented here as preferred formulation are only exemplary and are not intended to limit the scope of the invention in any way.

Preferred formulations for a hydrophilic antimigraine or antinausea drug comprise between about 0.01-10%, by weight, of the drug, about 60-90%, by weight, lipophilic carrier, between about 0.1-25%, by weight, mucoadhesive agent, between about 1-25%, by weight, sorption promoter and optionally a penetration enhancer or solubilizing agent, usually present in 1-30%, by weight.

Preferred formulations for the lipophilic drugs comprise between about 0.01-10%, by weight, of the drug, about 30-90%, by weight of hydrophilic carrier, between about 0.1-25%, by weight, of mucoadhesive agent, between 1 and 25% of sorption promoter and optionally between about 1-30%, by weight, solubilizing agent and/or permeation enhancer.

In one preferred embodiment of the invention, 0.01-10% of the drug is formulated with other components such as between about 60 to 90% by weight lipophilic carrier, between about 1.5 to 20% mucoadhesive agent, between about 10-20% of sorption promoter, between 0 to 30% solubilizing agent, and between about 1 to 30% permeation enhancer.

In another preferred embodiment of the invention, 0.01-10% drug is formulated in admixture with about 60 to 90%, by weight, of hydrophilic carrier, between about 1.5 and about 20% of mucoadhesive agent, between about 10 and 15% of sorption promoter and optionally between 0-30% of solubilizing agent and/or between about 1 and 30% of permeation enhancer.

In another preferred embodiment of the invention, the drug is formulated as a vaginal suppository which includes 0.01-10% of a hydrophilic drug, 75% of a lipophilic carrier SUPPOCIRE® AS2, 2% hydroxypropyl methylcellulose, and 15% of ethoxydiglycol (TRANSCUTOL®). The suppository may be a stand-alone device or be incorporated into a tampon or tampon-like device.

In another preferred embodiment of the invention, the drug is formulated as a vaginal suppository which includes 0.01-10% of a lipophilic drug, 75% of a hydrophilic carrier PEG 6000/PEG 1500, 2% hydroxypropyl methylcellulose, and 15% of ethoxydiglycol (TRANSCUTOL®). The suppository may be a stand-alone device or be incorporated into a tampon or tampon-like device.

C. Process for Formulating Hydrophilic or Lipophilic Drugs

The lipophilic or hydrophilic antimigraine or antinausea agents or inhibitors of membrane efflux system are formulated using the following process.

In a general method for preparing a formulation for a hydrophilic drug, the lipophilic carrier is melted at 45-50° C. in a heated vessel. The mucoadhesive agent is added to the carrier with stirring. The preferred hydrophilic drug is dissolved in the sorption promoter combined with the penetration enhancer and solublilizing agent. This mixture is added to the carrier/mucoadhesive agent suspension. The final formulation is poured into molds of the desired size and shape or incorporated into a device of the invention. The molds which are stored in a refrigerator at 4-6° C.

In a general method for preparing a formulation including a lipophilic drug, the hydrophilic carrier is melted in a heated vessel at an appropriate temperature recommended by manufacturer. The mucoadhesive agent is added to the carrier with stirring. The preferred lipophilic drug is dissolved in the sorption promoter, and penetration enhancer combined with the solublilizing agent are optionally added. This mixture is admixed with the carrier/mucoadhesive agent suspension. The final formulation is poured into molds of the desired size and shape or incorporated into a device of the invention. The final formulation is then placed in a refrigerator at 4-6° C.

D. Sustained Release

In one embodiment, the composition can be formulated as a sustained and controlled release drug system.

The antimigraine or antinausea drug which is formulated for controlled and sustained release is formulated either for continuous release or for pulsed delivery.

Continuous release or delivery means continuous and uninterrupted release of the drug from the formulation or device wherein the drug is formulated either in the matrix, microparticle, bioadhesive particle, liposomal suspension or any another system typically used for such release.

Pulsed release or delivery is a delivery of the drug in intermittent intervals. Such pulsed delivery may be provided, for example, by formulating the drug in the matrix, microparticle, bioadhesive particle, liposomal suspension or any another system, as described for continuous delivery, in individual layers interspaced with inactive layer of inactive, for example, dissolvable coatings or by using different formulating agents. Methods and formulating agents for sustained delivery are known in the art.

A drug delivery system for a controlled release must be capable of controlled release of a drug into the vaginal mucosa over several hours or more. This is achieved by the addition of time release additives such as hydrogel-forming polymers or non-errodible matrices, etc., known in the art.

Additionally, during the menstrual cycle when the pH of the vagina changes, the drug delivery systems additionally may contain buffers to stabilize pH to enhance absorption.

The sustained release composition of the invention is typically in a form of a cream, lotion, foam, suppository, tablet, microparticle, nanoparticle, capsule containing microparticles, liposomal suspension fluid, bioadhesive systems and microemulsions.

E. Bioadhesive Systems and Microemulsions

Bioadhesive microparticles or bioadhesive nanoparticles constitute still another intravaginal drug delivery system suitable for use in the present invention.

Bioadhesive systems and microemulsions are formulations particularly suitable for vaginal transmucosal delivery.

The microemulsion may contain pharmaceutically acceptable surfactants, for example, LABRASOL®, PLUROL® isostearate (Gattefossé), co-solvents such as isopropanol or ethanol, and water. Microemulsions containing one or more of the above components have been shown to improve bioavailability of antimigraine or antinausea drugs.

The bioadhesive systems use derivatives of cellulose such as hydroxypropyl cellulose and polyacrylic acid. They release the antimigraine or antinausea drugs for up to five days once they are placed in the appropriate formulation. This system represents a multi-phase liquid or semi-solid preparation which does not seep from the vagina as do most current suppository formulations. The microparticles or nanoparticles cling to the wall of the vagina and release the drug over a several hour period of time. Many of these systems were designed for nasal use, as described in U.S. Pat. No. 4,756,907, and 6,200,590 incorporated herein by reference, but can be easily modified for use in the vagina. The bioadhesive system may comprise microparticles or nanoparticles filled with the antimigraine or antinausea agent and may contain a surfactant for enhancing solubility and/or uptake of the drug. The microparticles have a diameter of 1-100 μm, whereas nanoparticles have a diameter of 10-1000 nm. Microparticles and nanoparticles can be prepared from starch, gelatin, albumin, collagen, or dextran according to methods known in the art.

Bioadhesive tablets are another drug delivery system suitable for transmucosal delivery. These bioadhesive systems use hydroxypropyl cellulose and polyacrylic acid. They release drugs for up to five days once they are placed in the appropriate formulation. The tablet of the invention has the shape of a suppository or a tampon so that the maximum contact is achieved between the vaginal wall and the tablet surface or such a shape as is suitable for incorporation into the device.

The drug formulated in a bioadhesive system may also be incorporated into creams, lotions, foams, paste, ointments, microemulsions, liposomal suspensions, and gels which can be applied to the vagina directly using an applicator or indirectly via a vaginal device. Processes for preparing pharmaceuticals in these vehicles can be found throughout the literature.

Suitable nontoxic pharmaceutically acceptable excipients for use in the compositions of the present invention will be apparent to those skilled in the art of pharmaceutical formulations and examples are described in REMINGTON: The Science and Practice of Pharmacy, 20^{th} Edition, A. R. Gennaro, ed., (2000). The choice of suitable carriers will depend on the exact nature of the particular vaginal dosage form desired, e.g., whether the antimigraine and/or antinausea agent is to be formulated into a cream, lotion, foam, ointment, paste, solution, microemulsions, liposomal suspension, microparticles, nanoparticles or gel, as well as on the physicochemical properties of the active ingredient(s).

Although the compositions described above typically contain only one pharmaceutically active ingredient from the group of antimigraine agents or antinausea for treatment of migraine or headache, nausea or vomiting, such compositions may additionally contain other pharmaceutical agents or a combination thereof, such as, for example, pain killers, antivirals, antipruritics, corticosteroids and other agents which may enhance the therapeutic effect of the primary drug.

All bioadhesive systems described above may be administered directly or via an intravaginal device.

IV. Device and/or System for Transvaginal Drug Delivery

The composition of the invention for transmucosal delivery is administered either directly to the vagina or is incorporated into the intravaginal device.

The intravaginal device of the invention is typically a tampon, tampon-like device, ring, pessary, strip, cup or foam which has a solid structure into which the formulation is incorporated and from which it is released in a timely fashion over a period of time. The time period is typically limited to from several minutes to 24 hours, preferably 4-8 hours, which is a hygienically acceptable time to leave the device in place.

Advantages of the medicated intravaginal device include continuous delivery of a predictable amount of the drug. The device may also have a washable and reusable design, such as, vaginal ring or pessary.

The intravaginal device for vaginal or transmucosal vaginal delivery of a antimigraine agent and/or antinausea agent is an intravaginal tampon, intravaginal ring, intravaginal pessary, intravaginal sponge, intravaginal tablet or intravaginal strip incorporated with a composition comprising a chemotherapeutic agent and/or inhibitor of membrane efflux systems formulated as a cream, lotion, foam, ointment, suppository, liposomal suspension, microemulsion, bioadhesive microparticle, bioadhesive nanoparticle, solution or gel.

The device may be configured for controlled release of the antimigraine or antinausea drugs where the drug incorporated into the device is formulated as a sustained release system, as described above.

In one embodiment, the invention provides a tampon device for delivering an antimigraine and/or antinausea agent across the vaginal mucosa comprising an absorbent vaginal tampon having a proximal end and a distal end. A cup-shaped porous foam portion at the distal end fits around the cervix of the systemic circulation and contains a pharmaceutical agent for delivery to the cervix. The device may also include a nonabsorbing axial tube having a distal opening and extending through the porous foam cup into the tampon for conducting blood flow to the absorbent material. Optionally, a retrieval string or tape connected to the tampon device is also included. The absorbent vaginal tampon is incorporated with a formulation containing any of the above-mentioned drugs or be coated with the formulation and used as a medicated tampon for individual drug or drug combination delivery.

In another embodiment of a tampon device, the distal porous foam cup has a rim which encircles the cervix. The rim has high concentrations of medication and is positioned away from the direct flow of blood which exudes from the cervix during menstruation.

In another embodiment of a tampon device, the distal porous foam cup has a rim which encircles the cervix. The rim has fingers extending into the fornix areas around the cervix and the tips of the fingers have high concentrations of medication and are positioned away from the direct flow of menstrual blood.

In another embodiment of a tampon device, a distal porous foam section is in the shape of a scoop, which only partially encircles the cervix. The porous foam scoop has a nib-like shape which is designed to wedge itself into the posterior fornix. The porous foam scoop is designed to deliver medication to the vaginal wall along the entire length of the porous foam scoop.

In another embodiment, a tampon device is sheathed in a thin, supple, non-porous material such as a plastic film or a coated gauze that surrounds the absorbent tampon material like a skirt and opens like an umbrella when it comes in contact with the vaginal environment. A drug incorporated into a strip, ideally suspended in a wax-like carrier that melts at body temperature, encircles the sheathed tampon. Contact with vaginal fluids or menstrual flow causes the tampon to swell, forcing the skirt to open like an umbrella and to press tightly against the vaginal wall, putting the drug in contact with the vaginal mucosa while effectively preventing the drug from being absorbed into the tampon.

In another embodiment of a tampon device, distal fibers of the tampon which contact the cervix have high concentrations of pharmaceutical agent for delivery of the agent to the cervical tissue.

In another embodiment of a tampon device, the tampon device has an outer tubing having perforations, wherein the outer tubing is concentric around an axial tube. The device has a distal porous foam section which in its dehydrated state is tight around the outer tubing. A bladder is located proximally to the porous foam and filled with liquid pharmaceutical agent. The bladder is connected to the outer tubing. An outer sheath covers the tampon. The sheath has an annular constriction distal to the bladder such that deployment of the tampon through the distal end of the sheath causes the liquid in the bladder to be forced out distally through the perforated outer tubing and into the porous foam.

In another embodiment of a tampon device, the tampon device has an annular delivery composition around the distal end. The composition contacts the vaginal mucosa for delivery of the antimigraine agent and/or antinausea agent. A non-absorbing axial tube opens distally and extends into the tampon for conducting blood flow to the absorbent material proximal to the porous foam. The annular composition can be a suppository, cream, ointment, foam, microparticles, paste, or gel.

Embodiments of the invention may include tampon devices of a standard length, or may be longer or shorter than standard tampons to facilitate positioning the tampon device closer to or in contact with the vaginal wall or with the cervix, depending on the location of tumor.

For purposes of simplifying the description of the invention and not by way of limitation, tampon or tampon-like devices, such as a suppository, for drug delivery will be described hereinafter, it being understood that all effective delivery systems are intended to be included within the scope of this invention and will be generally prepared in the same or similar manner.

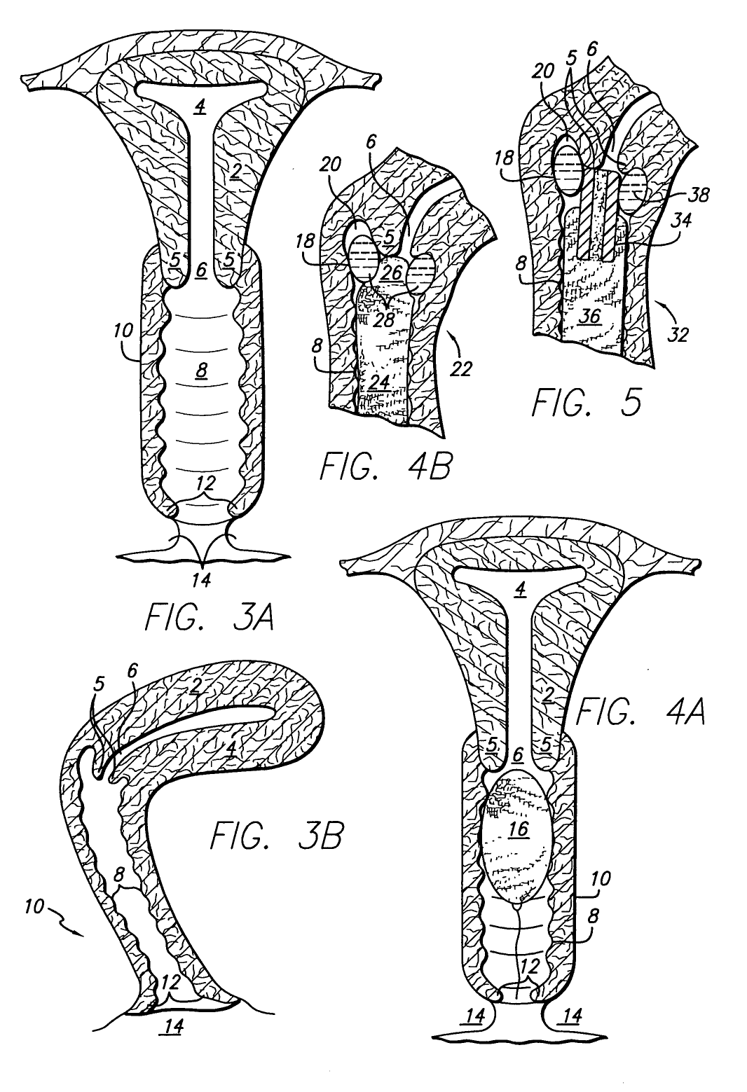

Particular device embodiments of the invention are described in greater detail in FIGS. 3-17. FIGS. 3A and 3B show anatomical arrangement of the vagina, systemic circulation and other organs. FIGS. 3-17 show various devices inserted into the vagina.

FIG. 3A is a cross-sectional representation of a portion of the female reproductive organs, including the uterus 2 and the vagina 8 in the upright orientation.

FIG. 3B is a cross-sectional side view representation thereof. The systemic circulation 2 is a muscular organ enclosing the womb 4, and opening at the cervix 5 via the cervical canal or cervical os 6. The vagina 8 is defined by a muscular tube 10 leading from the labia minora 12 and labia majora 14 to the cervix 5.

FIG. 4A is a cross-sectional representation of FIG. 3A showing placement of a drug delivery system 16 in the vagina 8 which drugs are introduced intravaginally to the vaginal wall 10 or transmucosally to the systemic circulation 2 by way of the vaginal blood vascular and lymphatic systems.

FIG. 4B is a cross-sectional representation of the vaginal area, adjacent the cervix 5, with a first embodiment of a tampon drug delivery system according to the invention. The tampon device 22 comprises an absorbent cylindrical tampon 24 comprised of fibrous material, for example cotton, having around its distal end 26 an annular delivery composition 28. The tampon device 22 places the annular delivery composition 28, supported around the distal end 26 of the tampon device 22, against the upper mucosa 18 of the vagina 8 and posterior fornix 20 for delivery through the vaginal surfaces in which the annular composition 28 is in contact. The annular composition 28 can be an annular suppository, foam, paste, gel, or any other formulation as described above, composed of suitable delivery components. The uterine discharge is absorbed by the tampon 24 and is prevented from carrying away the treatment composition.

FIGS. 5-12 depict various embodiments of devices of the invention which can be used to deliver an antimigraine or antinausea agent for treatment of reproductive organ cancers according to the invention.

The device is incorporated with a mucosal composition of the invention. Numerous methods exist by which a drug can be incorporated into the device. For example, the drug can be incorporated into a gel-like bioadhesive reservoir in the tip of the device. Alternatively, the drug can be in the form of a powdered material positioned at the tip of the tampon. The drug can also be absorbed into fibers at the tip of the tampon or other device, for example, by dissolving the drug in a pharmaceutically acceptable carrier and absorbing the drug solution into the tampon fibers. The drug can also be dissolved in a coating material which is applied to the tip of the tampon. This arrangement permits simultaneous drug delivery from the upper part of the device and absorption of the discharge or menstrual blood in the lower porous part of the tampon or tampon-like device. Alternatively, the drug can be incorporated into an insertable suppository, tablet, capsule, etc., which is placed in association with the tip of the tampon.

The tampon-like device can be constructed so as to improve drug delivery. For example, the tampon can be shaped to fit in the area of the posterior fornix and pubic symphysis and constructed so as to open up to have maximum surface area of contact for drug delivery. If the drug is in a reservoir on the surface of the device, the shape of the device should be such that it can maintain the reservoir towards a vaginal mucosal orientation for best predictable drug release characteristics.

The tampon device can also be constructed so as to have a variable absorption profile. For example, the drug area at the tip of the tampon device could be different from that of the more proximal area in order to force the drug to diffuse out into tissue, as opposed to down into the absorbent part of the tampon. Alternatively, there could be a non-absorbing channel around the cervix for the first centimeter or so in order to minimize menstrual flow from washing away the drug composition.

The release of drug from the tampon device should be timed to provide proper systemic concentration of the drug over a typical length of use of a tampon device, usually 1-8 hours.

FIG. 5 is a cross-sectional representation of the vaginal area adjacent the cervix 5 with a second embodiment of a tampon drug delivery system according to the invention. In this embodiment, tampon device 32 includes a non-porous tube 34 which communicates with the cervical os 6 for delivery of the menstrual discharge from the cervical os to an absorbent cylindrical tampon 36 comprised of fibers, for example cotton, for absorbing the discharge. The tube 34 prevents contact of the discharge with an annular drug delivery composition 38.

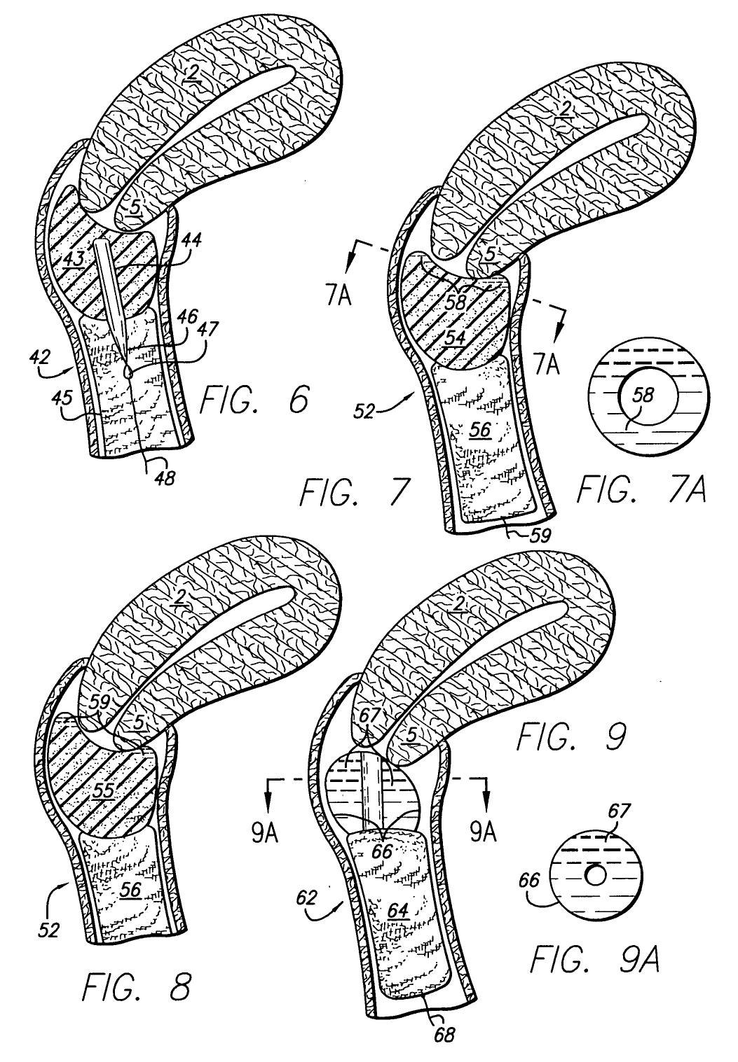

FIG. 6 is a cross-sectional representation of the vaginal area adjacent the cervix 5 with a third embodiment of a tampon drug delivery system according to the invention. In FIG. 6, the tampon device 42 includes a distal porous foam section 43 which is in the shape of a cup in the expanded state. In the center of the porous foam section 43 is a non-porous tube 44 which will conduct blood flow to absorbent tampon 45 proximal to the porous foam section 43. The porous foam is preferably a soft, light weight, physiologically inert foam material of pqlyurethane, polyester, polyether, such as described in U.S. Pat. No. 4,309,997, or other material such as collagen as described in U.S. Pat. No. 5,201,326, both incorporated herein by reference. The axial tube is preferably a non-absorptive physiologically inert material, such as rubber or plastic, and can be coated on its inner surface with an anticoagulant. The proximal end 46 of the tube 44 has a plastic loop 47 to which a string 48 may be tied for removal of the tampon device 42. The cup-shaped porous foam section 43 fits around the cervix 5 of the systemic circulation 2 and contains antineoplastic drug which may be delivered to the cervical tissue.

FIG. 7 is a cross-sectional representation of the vaginal area adjacent the cervix 5 with a fourth embodiment of a tampon drug delivery system according to the invention. In FIG. 7, the tampon device 52 includes a distal porous foam cup 54 and a proximal absorbent tampon 56. The porous foam cup 54 has a rim 58 which encircles the cervix 5, and which contains high concentrations of an antimigraine and/or antinausea agent. The rim 58 area of the porous foam cup 54 is away from the direct flow of blood. The tampon device 52 includes a string 59 for removal of the tampon device 52. FIG. 7A is a cross-sectional view of the embodiment shown in FIG. 7, taken in the direction indicated by the arrows labeled 7A in FIG. 7. As illustrated in FIG. 7A, the rim 58 area forms a ring which contains a high concentration of the drug. Alternatively, as illustrated in FIG. 8, the entire porous foam cup 55 may contain the drug composition, not just in the ringed tip area 59 near the cervix 5.

FIG. 9 is a cross-sectional representation of the vaginal area adjacent the cervix 5 with a fifth embodiment of a tampon drug delivery system according to the invention. In FIG. 9, the tampon device 62 includes a proximal absorbent tampon 64 and a distal section 66 which includes a dissolvable suppository or gel capsule 67 filled with liquid composition of the invention. The device, prior to the medication dissolution or release has a “doughnut” shape to allow for blood to pass through the center of the tampon 64. The tampon device 62 includes a string 68 attached to the tampon 64 for removal of the tampon device 62. FIG. 9A is a cross-sectional view of the of the embodiment shown in FIG. 9, taken in the direction indicated by the arrows labeled 9A in FIG. 9, and illustrates the doughnut shape of the medication filled suppository or gel capsule 67.

FIG. 10 is a cross-sectional representation of the vaginal area adjacent the cervix 5 with a sixth embodiment of a tampon drug delivery system according to the invention. In FIG. 10, the tampon device 72 includes a porous foam distal section 74 which is in the shape of a cup with “fingers” 76 which extend into the fornix areas 20 around the cervix 5. The tips of the fingers 76 contain high concentrations of medication which may be delivered to areas away from the direct flow of blood or discharge as the blood or discharge moves into absorbent tampon 78 proximal to the cup-shaped porous foam distal section 74. The tampon device 72 includes a string 79 for removal of the tampon device 72. FIG. 10A is a side view of the porous foam cup 74 and illustrates the fingers 76 which extend into the fornix areas 20 around the cervix 5 (FIG. 10).

It will be readily apparent to a person skilled in the art that the characterization of the drug delivery device as having an annular shape is only an approximate description of the shape formed by fluid or semisolid drug delivery devices positioned around a cylinder and in contact with adjacent vaginal wall mucosa, and all shapes which conform to the vaginal mucosa and external cervical surfaces are intended to be included within and indicated by the term “annular”. Moreover, use of the term “annular” does not restrict the invention to the use of such devices which encircle the entire cervix (i.e. 360°). Devices which span an angle of less than 360°, but which make sufficient contact with the vaginal mucosa to deliver sufficient quantity of the drug are within the scope of the invention.

The annular drug delivery composition (FIGS. 4 or 5) can be an absorbent material which expands in the presence of fluid or body heat to completely fill the space between the tampon 22, 32 and the vaginal mucosa 18.

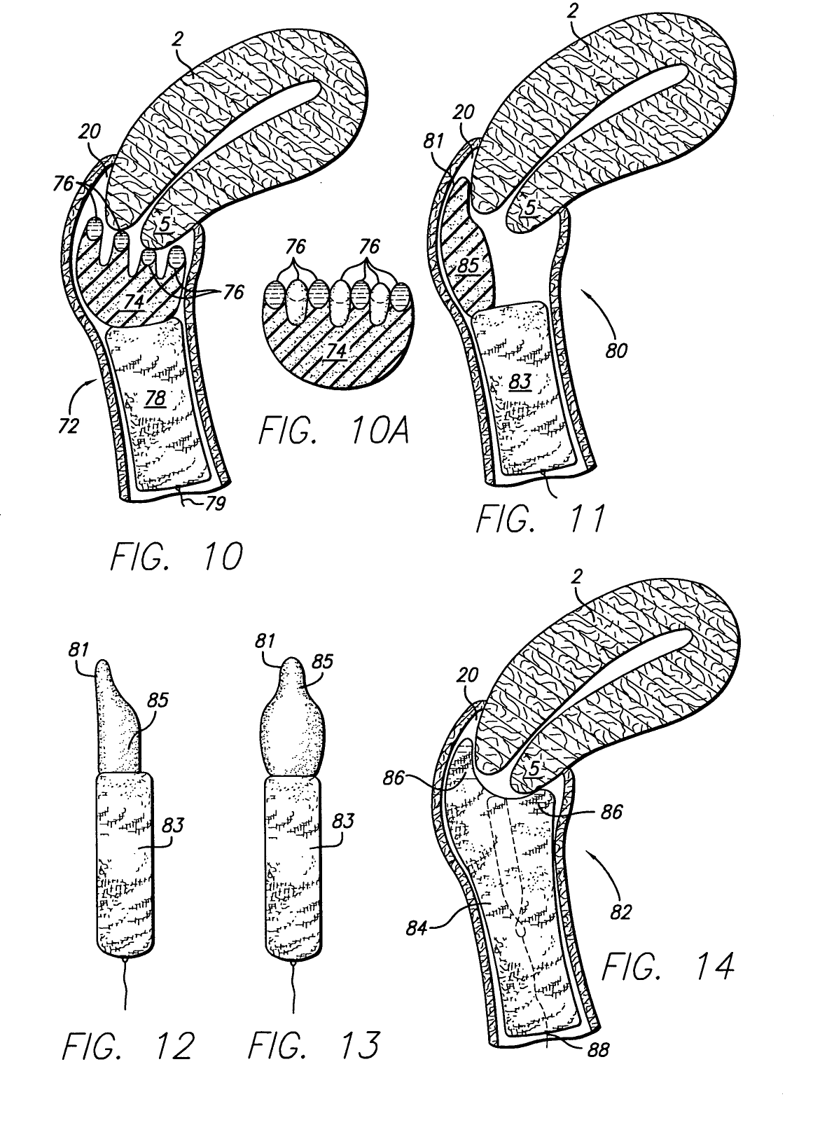

FIG. 11 illustrates such a drug delivery device having an annular shape which does not completely encircle the entire cervix. FIG. 11 is the representation of FIG. 2 showing placement of a seventh embodiment of a tampon device 80 incorporating a scoop-shaped porous foam section 85. FIG. 12 is a side view of the tampon device 80 and FIG. 13 is a front view of the tampon device 80. The scoop-shaped porous foam section 85 is annular in shape, but does not completely encircle the cervix 5. Instead, the scoop-shaped porous foam section has a nib-shaped tip 81 which is designed to wedge itself into the posterior fornix 20. The scoop-shaped porous foam section 85 is designed to deliver medication to the vaginal wall along the entire length of the scoop-shaped porous foam section 85.

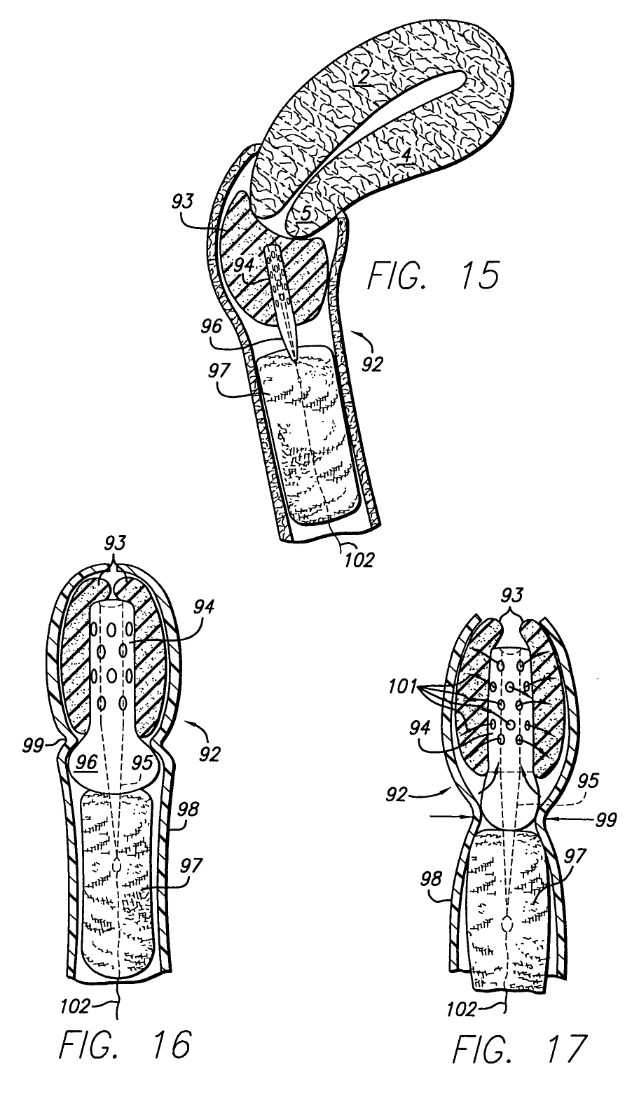

FIG. 14 is a cross-sectional representation of the vaginal area adjacent the cervix 5 with an eighth embodiment of a tampon drug delivery system according to the invention. In FIG. 14, the tampon device 82 comprises an absorbent tampon 84. The section 86 of the tampon 84 which rests against the cervix 5 contains high concentrations of medication. As the fibers absorb fluid, the tampon 84 expands around the cervix 5 and delivers medication to the tissue. The blood will be drawn to proximal sections of the tampon 84 as fibers become more absorbent in this area. The tampon device 82 includes a string 88 for removal of the tampon device 82.

Suitable cylindrical cartridge containers or inserter tubes which assist in the insertion and storage of the tampon systems of the present invention will be apparent to those skilled in the art of tampon construction. Examples are described in U.S. Pat. Nos. 4,3178,447; 3,884,233; and 3,902,493, incorporated herein by reference.

In general practice, a drug delivery device as described herein is placed into the vagina and the inserter tube is removed. The device, such as a tampon, contacts the inner wall of the vagina where the mucoadhesive agents facilitate adhesion of the drug released from the device to the vaginal wall where it is therapeutically effective.

FIG. 15 is a cross-sectional representation of the vaginal area adjacent the cervix 5 with another embodiment of a tampon drug delivery system according to the invention. In FIG. 15, the tampon device 92 includes a distal porous foam section 93 which, in its dehydrated, sheathed state (FIG. 16), is tight around a perforated outer tube 94. The perforated outer tube 94 is connected to a bladder 96 located proximally which is filled, for example, with liquid medication. Within the perforated outer tube 94 is a concentric inner tube 95 which provides a pathway for blood to flow into an absorbent tampon 97 which is proximal to the porous foam section 93. Prior to insertion, the tampon device 92 is enveloped in a sheath 98 which is necked down at site 99 between the porous foam section 93 and the bladder 96 so that, when the tampon device 92 is deployed and the sheath 98 moves over the bladder 96, the medication is forced out seen as 101, through the perforated outer tube 94 into the porous foam section 93 (FIG. 17). The tampon device 92 includes a string 102 for removal of the tampon device 92.

Another example of a suitable controlled release drug delivery system for the present invention is the vaginal ring. Vaginal rings usually consist of an inert elastomer ring coated by another layer of elastomer containing the drug to be delivered. The rings can be easily inserted, left in place for the desired period of time, up to 7 days, then removed by the user. The ring may be solid or hollow containing the antimigraine and/or antinausea drug or it may be a porous material releasing the drug therefrom. The ring can optionally include a third, outer, rate-controlling elastomer layer which contains no drug. Optionally, the third ring can contain a second drug for a dual release ring. The drug can be incorporated into polyethylene glycol throughout the silicone elastomer ring to act as a reservoir for drug to be delivered.

Pessaries, cups, strips, tablets and suppositories are other examples of drug delivery systems which can be used in the present invention. These systems have been previously used for delivery of vaginal contraceptives, and have been described extensively in the literature.

Another example of a delivery system is the vaginal sponge, film and foam. The desired pharmaceutical agent can be incorporated into a silicone matrix which is coated onto a cylindrical drug-free pqlyurethane vaginal sponge, as described in the literature.

In practice, the drug delivery system, that is a composition or a device of the invention, are applied or administered upon diagnosis of migraine or nausea. Typically, the treatment is continued for as long as needed to treat the headache, nausea or vomiting, to maintain state or prevent further growth.

### EXAMPLE 1

Preparation of Sumatriptan Vaginal Suppository

This example describes a process for preparation of intravaginal suppositories.

The dose of sumatriptan (Global Trade Alliance, Scottsdale, Ariz.) was 20 mg. Vaginal suppositories were formulated and prepared 24 hours prior to administration. The four basic ingredients for the suppositories were distilled water (15% wt), SUPPOCIRE AS2X (Gattefossé, Westwood, N.J.) (67.5% wt), hydroxypropyl methylcellulose (HPMC) (obtained as METHOCEL® K, HPMC K15M, from Dow Chemical, Midland, Mich.) (1.5% wt), a mucoadhesive agent, and TRANSCUTOL® (Gattefossé) (15% wt), a permeation enhancer.

To make eight suppositories, 10.8 grams of SUPPOCIRE, 240 mg of HPMC, 2.4 grams of TRANSCUTOL, and the calculated dose of the drug were weighed out. SUPPOCIRE was melted in a disposable 100 mL polypropylene beaker suspended in a water bath at 50° C. The solution was stirred until completely melted. HPMC and TRANSCUTOL were then added and mixed. Finally, the drug was added combined with 2.4 grams of distilled water. After sufficient mixing, the warm suppository mass was quickly poured into commercial nickel-plated brass suppository molds available from the Adelphi Group of Company (West Sussex, UK). Suppositories were kept refrigerated until use.

### EXAMPLE 2

Preparation of Metoclopramide Vaginal Suppository

This example describes the preparation of metoclopramide-containing vaginal suppositories.

Metoclopramide hydrochloride is commercially obtained from ICN Biomedicals, Inc. (Costa Mesa, Calif.). Vaginal suppositories comprising a dose of 50 mg per suppository were prepared using the method identical to the procedure described for sumatriptan suppositories. The composition of the pharmaceutical excipients in these formulations was SUPPOCIRE AS2X (66% wt), HPMC (1.5% wt), TRANSCUTOL (15% wt), and distilled water (15% wt).

Suppositories comprising other antimigraine or antinausea drugs are prepared the same way except that their amount, including of excipients, may vary.

### EXAMPLE 3

Preparation of Diclofenac Sodium Vaginal Suppository

This example describes the procedure for preparation of hydrophilic diclofenac vaginal suppositories.

A binary mixture of 7.18 grams of polyethylene glycol (PEG) 3350 and 3.86 grams of PEG 6000 (Fisher Scientific, Pittsburgh, Pa.) is melted on a water bath. To the homogenous PEG solution 400 mg of triethanolamine (Sigma/Aldrich, St. Louis, Mo.) is added. In a separate container, 400 mg diclofenac sodium (Spectrum Chemicals & Laboratory Products, Gardena, Calif.) is dissolved in 2.4 grams of TRANSCUTOL that is further diluted with 2.4 grams of distilled water. Both solutions are combined and cooled under stirring. After reaching suitable viscosity, aliquots of the suppository mass are filled into nickel-plated brass molds.

### EXAMPLE 4

Preparation of Promethazine Vaginal Film

This example describes the process for preparation of vaginal film composition.

In a 100 mg glass beaker, 240 mg promethazine hydrochloride (Spectrum Chemicals & Laboratory Products, Gardena, Calif.) is dissolved in 2 grams of distilled water and 1.5 grams of TRANSCUTOL. This drug solution is combined with a polymeric algininc acid solution consisting of 500 mg alginic acid, sodium salt (CarboMer, Inc., Westborough, Mass.) and 8 grams of water. Thin films of approximately 1 mm in thickness will be prepared using a hand-operated CAMAG TLC plate coater (CAMAG Scientific, Inc., Wilmington, N.C.).

### EXAMPLE 5

Preparation of Metoclopramide Vaginal Foam

This example describes the preparation of a medicated vaginal foam.

Metoclopramide hydrochloride (ICN Biomedicals, Inc., Costa Mesa, Calif.) is dissolved in a mixture of PEG 400 (10% wt, Fisher Scientific, Pittsburgh, Pa.) and TRANSCUTOL (15% wt). In a separate container 4.5% (wt) alginic acid, sodium salt is dissolved in distilled water (70% wt). Both solutions are combined and aliquots of 5 mL filled into plastic syringe. Following a thorough freezing process at −80° C., the samples were removed from the syringe mold and lyophilized to form the medicated vaginal foam.

What is claimed is:

1. A mucoadhesive composition suitable for a mucosal or transmucosal vaginal delivery of an anti-migraine or antinausea drug, said composition comprising from about 0.001 to about 3000 mg of the antimigraine or antinausea drug, from about 30 to about 95% of a lipophilic or hydrophilic carrier, from about 0.1 to about 25% of a muccadhesive agent and from about 5 to about 30% of a sorption promoter.

2. The composition of claim 1 wherein said mucoadhesive agent is hydroxypropyl methylcellulose, a cellulose derivative, a natural gum, alginate or pectin, present in from about 1.5 to about 15%, by weight, wherein said sorption promoter is ethoxydiglycol, polyethylene glycol caprylic/capric glycerides, a glycol derivative with oleic acid esters of propylene glycol and glycerol or interesterified stone oil present in from about 2 to about 30%, by weight, wherein the lipophilic carrier is a saturated mono-, di- or triglyceride of fatty acids having carbon chain of from 8 to 18 carbons, or a mixture thereof, present from about 30 to about 95%, by weight, wherein the hydrophilic carrier is a polyethylene glycols (PEG) of a molecular weight between about 200 and 8000, or a derivative or mixture thereof, PEG 6000/PEG 1500, PEG 6000/PEG 1500/PEG 400, or PEG 6000/PEG 400, or PEG 8000/PEG 1500, present from about 30 to about 95%, by weight.

3. The composition of claim 2 wherein said mucoadhesive agent is hydroxypropyl methylcellulose present in from about 1.5 to about 5%, wherein said sorption promoter is ethoxydiglycol present in from about 15%, wherein said lipophilic carrier is the saturated mono-, di- or triglyceride of fatty acids having carbon chain of from 8 to 18 carbons and a mixture thereof, present from about 65 to about 70%, and wherein said composition further comprises from about 1 to 1000 mg of antimigraine or antinausea drug.

4. The composition of claim 3 wherein said antimigraine drug is selected from the group of compounds consisting of ergotamine, dihydroergotamine, ergostine, butalbital, phenobarbital, acetaminophen, diclofenac sodium, ketoprofen, ketorolac, ibuprofen, prioxicam, naproxen, acetylsalicilic acid, flurbiprofen, tolfenamic acid, butorphanol, meperidine, methadone, sumatriptan, naratriptan, razatriptan, zolmitriptan, almotriptan, eletriptan, dexamethasone, hydrocortisone, isometheptene, chlorpromazine, diazepam, droperidol, valproic acid, gabapentin, topiramate and divalproex sodium, and wherein said antinausea drug is selected from the group consisting of metoclopramide, prochlorperazine, domperidone, ondansetron, tropisetron, dolasetron, nabilone, dronabinol, levonantradol, aprepitant, cyclizine, promethazine and combination thereof.

5. The composition of claim 4 wherein said antimigraine or antinausea drug is ergotamine administered in a range from about 15 to about 300 mg/day, diclofenac sodium administered in a range from about 100 to about 500 mg/day, sumatriptan administered in a range from about 20 to 500 mg/day, zolmitriptan administered in a range from about 10 to about 420 mg/day, naratriptan administered in a range from about 10 to about 350 mg/day, metoclopramide administered in a range from about 20 to 120 mg/dose, prochlorperazine administered in a range from about 25 to 150 mg/dose, ondansetron administered in a range from about 30 to 210 mg/dose, dronabinol administered in a range from about 10 to 50 mg/day, and promethazine administered from about 12 to about 80 mg/dose.

6. A method for treatment of migraine and headache, nausea or vomiting associated with chemotherapy, radiotherapy, surgery, pregnancy or menopause, said method comprising a step:
administering to a female subject in need of such treatment vaginally a mucoadhesive composition comprising from about 0.001 to about 3000 mg of the antimigraine or antinausea drug, from about 30 to about 95% of a lipophilic or hydrophilic carrier, from about 0.1 to about 25% of a mucoadhesive agent and from about 5 to about 30% of a sorption promoter,
wherein said antimigraine drug is selected from the group of compounds consisting of ergotamine, dihydroergotamine, ergostine, butalbital, phenobarbital, acetaminophen, diclofenac sodium, ketoprofen, ketorolac, ibuprofen, prioxicam, naproxen, acetylsalicilic acid, flurbiprofen, tolfenamic acid, butorphanol, meperidine, methadone, sumatriptan, naratriptan, razatriptan, zolmitriptan, almotriptan, eletriptan, dexamethasone, hydrocortisone, isometheptene, chlorpromazine, diazepam, droperidol, valproic acid, gabapentin, topiramate and divalproex sodium, and wherein said antinausea drug is selected from the group consisting of metoclopramide, prochlorperazine, domperidone, ondansetron, tropisetron, dolasetron, nabilone, dronabinol, levonantradol, aprepitant, cyclizine, promethazine, and a combination thereof.

7. The method of claim 6 wherein said composition is formulated and administered vaginally as a cream, lotion, foam, film, suppository, tablet, microparticle, nanoparticle, capsule, capsule containing microparticles, emulsion, liposomal suspension fluid, a bioadhesive system or microemulsion.

8. The method of claim 7 wherein said composition formulated as a cream, lotion, foam, film, suppository, tablet, microparticle, nanoparticle, capsule, capsule containing microparticles, emulsion, liposomal suspension fluid, a bioadhesive system or microemulsion is incorporated into an intravaginal device and said device is inserted vaginally.

9. The method of claim 8 wherein said composition is administered at the onset of the migraine or during nausea.

10. The method of claim 9 for treatment of migraine wherein said treatment comprises administration of a composition comprising ergotamine from about 15 to about 300 mg/day, diclofenac sodium from about 100 to about 500 mg/day, sumatriptan from about 20 to 500 mg/day, zolmitriptan from about 10 to about 420 mg/day or naratriptan from about 10 to about 350 mg/day.

11. The method of claim 9 for treatment of nausea wherein said treatment comprises administration of a composition comprising metoclopramide from about 20 to 120 mg/dose, prochlorperazine from about 25 to 150 mg/dose, ondansetron from about 30 to 210 mg/dose, dronabinol from about 10 to 50 mg/day or promethazine from about 12 to about 80 mg/dose.

12. The method of claim 8 wherein said intravaginal device is a tampon, tampon-like device, pessary, ring, tablet, capsule, pad, patch, suppository, cup, sponge, strip, foam or intravaginal iontophoretic system, and wherein said antimigraine or antinausea drug is released form said device.

13. The method of claim 12 wherein said device is inserted into the vagina at the onset of the migraine or during nausea.

14. The method of claim 13 wherein said device is the tampon.

15. The method of claim 14 for treatment of migraine wherein said treatment comprises administration of a composition comprising ergotamine from about 15 to about 300 mg/day, diclofenac sodium from about 100 to about 500 mg/day, sumatriptan from about 20 to 500 mg/day, zolmitriptan from about 10 to about 420 mg/day or naratriptan from about 10 to about 350 mg/day.

16. The method of claim 14 for treatment of nausea wherein said treatment comprises administration of a composition comprising metoclopramide from about 20 to 120 mg/dose, prochlorperazine from about 25 to 150 mg/dose, ondansetron from about 30 to 210 mg/dose, dronabinol from about 10 to 50 mg/day or promethazine from about 12 to about 80 mg/dose.

17. An intravaginal device for mucosal and transmucosal delivery of an antimigraine or antinausea drug
wherein said device is a tampon, tampon-like device, pessary, patch, ring, tablet, pad, suppository, sponge, film, foam, strip or intravaginal iontophoretic system incorporated with a mucoadhesive composition comprising antimigraine drug, antinausea drug or a combination thereof;
wherein said device is incorporated with a mucoadhesive composition comprising from about 0.001 to about 3000 mg of the antimigraine or antinausea drug, from about 30 to about 95% of a lipophilic or hydrophilic carrier, from about 0.1 to about 25% of a mucoadhesive agent and from about 5 to about 30% of a sorption promoter,
wherein said antimigraine drug is selected from the group of compounds consisting of ergotamine, dihydroergotamine, ergostine, butalbital, phenobarbital, acetaminophen, diclofenac sodium, ketoprofen, ketorolac, ibuprofen, prioxicam, naproxen, acetylsalicilic acid, flurbiprofen, tolfenamic acid, butorphanol, meperidine, methadone, sumatriptan, naratriptan, razatriptan, zolmitriptan, almotriptan, eletriptan, dexamethasone, hydrocortisone, isometheptene, chlorpromazine, diazepam, droperidol, valproic acid, gabapentin, topiramate and divalproex sodium, and wherein said antinausea drug is selected from the group consisting of metoclopramide, prochlorperazine, domperidone, ondansetron, tropisetron, dolasetron, nabilone, dronabinol, levonantradol, aprepitant, cyclizine, and promethazine, each alone or in combination with an another pharmaceutical agent or a pharmaceutically acceptable excipient; and
wherein said antimigraine or antinausea drug is released from said device following the insertion of the device into vagina.

18. The device of claim 17 wherein the device is the tampon and said composition is incorporated withing the tampon.

19. The device of claim 17 wherein the device is the tampon and said composition is applied as a coating on the surface of the tampon.

20. The device of claim 17 wherein the device is the tampon and said composition is formulated as a foam or film and is either incorporated into the tampon or applied to the surface of the tampon.
